(19) United States
(12) Patent Application Publication
(10) Pub. No.: US 2010/0201797 A1
(43) Pub. Date: Aug. 12, 2010

(54) ENDOSCOPE DEVICE AND METHOD FOR DRIVING ENDOSCOPE DEVICE

(75) Inventors: Makoto SHIZUKUISHI, Kanagawa (JP); Masami HATORI, Kanagawa (JP); Yoshiaki ISHIMARU, Kanagawa (JP)

Correspondence Address: Studebaker & Brackett PC, One Fountain Square, 11911 Freedom Drive, Suite 750, Reston, VA, 20190, US

(73) Assignee: FUJIFILM CORPORATION, Tokyo (JP)

(21) Appl. No.: 12/696,416

(22) Filed: Jan. 29, 2010

(30) Foreign Application Priority Data
Jan. 30, 2009 (JP) 2009-019949
May 27, 2009 (JP) 2009-127952

(51) Int. Cl. H04N 7/18; A61B 1/04

(52) U.S. Cl. 348/68; 600/109; 348/E07.085

(57) ABSTRACT

An endoscope device includes: a light source including an LED for emitting an R light, an LED for emitting a G light and an LED for emitting a B light, and a solid-state image pick-up element having a plurality of pixel parts including a photoelectric conversion part that may receive the R light, the G light and the B light to generate electric charges corresponding to the received lights and floating gates that may selectively store the electric charges generated in the photoelectric conversion part and a reading circuit that independently reads signals corresponding to the electric charges respectively stored in the floating gates.

### CROSS-REFERENCE TO RELATED APPLICATION

This application is based on and claims priority under 35 USC 119 from Japanese Patent Application No. 2009-019949 filed on Jan. 30, 2009 and Japanese Patent Application No. 2009-127952 filed on May 27, 2009.

### BACKGROUND

1. Technical Field

The present invention relates to an endoscope device provided with a solid-state image pick-up element having a plurality of pixel parts and a method for driving the endoscope device.

2. Related Art

An electronic endoscope device includes a surface sequential system in which an object is lighted sequentially by the lights of colors of R (red color), G(green color) and B(blue color) respectively and the lights from the object are received by a solid-state image pick-up element having no color filter to carry out a shooting operation or an image recording operation and a surface simultaneous system in which an object is lighted by a white color light and the lights from the object are received by a solid-state image pick-up element on which an RGB color filter is mounted to carry out a shooting operation or an image recording operation.

Since the electronic endoscope device of the surface sequential system may obtain color signals of RGB respectively from the pixels of the solid-state image pick-up element, a signal interpolating process is not necessary and a false color is hardly generated. Further, since the color filter is not provided, color reproducibility may be determined by the color characteristics (spectral characteristics of the colors of RGB respectively) of a light source, an image of high fidelity may be obtained. Further, since the color filter is not mounted, the solid-state image pick-up element may be miniaturized and a diameter of the endoscope may be reduced. Thus, a burden to a patient may be reduced. As described above, the surface sequential system has various advantages, so that the surface sequential system is available to improve a diagnostic accuracy due to an improvement of an image quality or reduce the burden of the patient due to the reduced diameter of the endoscope.

A lighting system of an object to be shot or recorded in the surface sequential system includes a system in which a light source for emitting a white light is combined with a color filter for transmitting the color lights of RGB respectively and a system using three light sources for emitting an R light, a G light and a B light respectively. The system of the latter is disclosed in, for instance, JP-A-2007-275243.

However, since the surface sequential system includes steps of, for instance, emitting the R light, reading a signal, emitting the G light, reading a signal, emitting the B light and reading a signal, a signal reading period is long. Accordingly, it takes long time until a next light is emitted. Thus, there is a fear that the object to be shot or recorded may move with high possibility during this period and a color divergence may occur to deteriorate an image quality. When the signal reading period is shortened, the color divergence may be suppressed. However, when a signal reading operation is carried out at high speed, an exposure time of each pixel needs to be shortened, so that the deterioration of sensitivity cannot be avoided. Further, in accordance with the high-speed operation, a quantity of heat generation of an element itself is undesirably increased. When the pixels are increased, the signal reading period is naturally lengthened. Thus, the deterioration of the image quality is more requested to be suppressed hereafter.

### SUMMARY

The present invention is proposed by considering the above-described circumstances and it is an object of the present invention to provide an endoscope device that may prevent a color divergence and improve an image quality and a method for driving the endoscope device.

An endoscope device of the present invention includes: a light source that may independently emit a first light, a second light and a third light; and a solid-state image pick-up element having a plurality of pixel parts including a photoelectric conversion part that may receive the first light, the second light and the third light to generate electric charges corresponding to the received lights and a plurality of electric charge storage parts that may selectively store the electric charges generated in the photoelectric conversion part, and a signal reading part that independently reads signals corresponding to the electric charges respectively stored in the plurality of electric charge storage parts.

A method for driving an endoscope device of the present invention includes a sold-state image pick-up element having a plurality of pixel parts, the plurality of pixel parts including a photoelectric conversion part that may receive lights incident from an object to be shot or recorded to generate electric charges corresponding to the received lights and a first electric charge storage part, a second electric charge storage part and a third electric charge storage part that may selectively store the electric charges generated in the photoelectric conversion part. The method for driving an endoscope device includes: a first driving step that emits a first light to store in the first electric charge storage part the electric charge generated in the photoelectric conversion part by the light incident from the object to be shot or recorded relative to the first light; a second driving step that emits a second light to store in the second electric charge storage part the electric charge generated in the photoelectric conversion part by the light incident from the object to be shot or recorded relative to the second light; a third driving step that emits a third light to store in the third electric charge storage part the electric charge generated in the photoelectric conversion part by the light incident from the object to be shot or recorded relative to the third light and a signal reading step that reads signals corresponding to the electric charges respectively stored in the first electric charge storage part, the second electric charge storage part and the third electric charge storage part after the first driving step, the second driving step and the third driving step are finished.

A method for driving an endoscope device of the present invention includes a sold-state image pick-up element having a plurality of pixel parts, the plurality of pixel parts including a photoelectric conversion part that may receive lights incident from an object to be shot or recorded to generate electric charges corresponding to the received lights and a first electric charge storage part, a second electric charge storage part and a third electric charge storage part that may selectively store the electric charges generated in the photoelectric conversion part. The method for driving an endoscope device includes a first driving step that emits a G light, a B light and an R light at the same time or continuously to store in the first electric charge storage part the electric charge generated in the photoelectric conversion part by the light incident from the object to be shot or recorded relative to the emitted lights; a second driving step that emits the B light to store in the second electric charge storage part the electric charge generated in the photoelectric conversion part by the light incident from the object to be shot or recorded relative to the B light; a third driving step that emits the R light to store in the third electric charge storage part the electric charge generated in the photoelectric conversion part by the light incident from the object to be shot or recorded relative to the R light; a signal reading step that reads signals corresponding to the electric charges respectively stored in the first electric charge storage part, the second electric charge storage part and the third electric charge storage part after the first driving step, the second driving step and the third driving step are finished; and a color difference signal generating step that forms a first color difference signal from the signal read from the first electric charge storage part and the signal read from the second electric charge storage part and generates a second color difference signal from the signal read from the first electric charge storage part and the signal read from the third electric charge storage part.

A method for driving an endoscope device of the present invention includes a solid-state image pick-up element having a plurality of pixel parts, the plurality of pixel parts including a photoelectric conversion part that may receive lights incident from an object to be shot or recorded to generate electric charges corresponding to the received lights and a first electric charge storage part and a second electric charge storage part that may selectively store the electric charges generated in the photoelectric conversion part. The method for driving an endoscope device includes: a first driving step that emits a first light to store in the first electric charge storage parts of all the pixel parts the electric charges generated in the photoelectric conversion part by the light incident from the object to be shot or recorded relative to the first light; a second driving step that emits a second light to store in the second electric charge storage parts of the pixel parts half as many as the plurality of pixel parts the electric charges generated in the photoelectric conversion part by the light incident from the object to be shot or recorded relative to the second light; a third driving step that emits a third light to store in the second electric charge storage parts of the remaining pixel parts half as many as the plurality of pixel parts the electric charges generated in the photoelectric conversion part by the light incident from the object to be shot or recorded relative to the third light; a signal reading step that reads the signals corresponding to the electric charges respectively stored in the first electric charge storage parts and the second electric charge storage parts after the first driving step, the second driving step and the third driving step are finished and an interpolating step that interpolates the signal corresponding to the second light or the signal corresponding to the third light that is not obtained from the pixel parts by using a signal corresponding to the second light and a signal corresponding to the third light that are obtained from pixel parts in the periphery of the pixel parts.

According to the present invention, an endoscope device that may prevent a color divergence and improve an image quality and a method for driving the endoscope device may be provided.

### BRIEF DESCRIPTION OF THE DRAWINGS

FIG. 1 is a diagram showing a schematic structure of an endoscope device for explaining one exemplary embodiment of the present invention;

FIGS. 2A and 2B are diagrams showing a schematic structure of a solid-state image pick-up element in FIG. 1;

FIG. 3 is an equivalent circuit diagram of an inner structure of one pixel part 100 shown in FIG. 2;

FIG. 4 is a schematic plan view showing a layout example of the pixel part 100 based on the equivalent circuit diagram shown in FIG. 3;

FIG. 5 is a timing chart for explaining an operation of the endoscope device shown in FIG. 1;

FIG. 6 is a schematic diagram for explaining an electric charge storing operation of the endoscope device shown in FIG. 1;

FIG. 7 is a diagram for explaining the effect of the endoscope device shown in FIG. 1;

FIG. 8 is a timing chart for explaining an operation of a first modified example of the endoscope device shown in FIG. 1;

FIG. 9 is a schematic diagram for explaining an electric charge storing operation of the first modified example of the endoscope device shown in FIG. 1;

FIG. 10 is a diagram showing a second modified example of the endoscope device shown in FIG. 1;

FIG. 11 is a diagram showing a relation between the spectral characteristics of a color filter and a bright line of a special light;

FIG. 12 is a timing chart for explaining an operation of the second modified example of the endoscope device shown in FIG. 1;

FIG. 13 is a schematic diagram for explaining an electric charge storing operation of the second modified example of the endoscope device shown in FIG. 1;

FIG. 14 is a diagram showing a third modified example of the endoscope device shown in FIG. 1;

FIG. 15 is a timing chart for explaining an operation of the third modified example of the endoscope device shown in FIG. 1;

FIG. 16 is a schematic diagram for explaining an electric charge storing operation of the third modified example of the endoscope device shown in FIG. 1;

FIG. 17 is a diagram showing a fourth modified example of the endoscope device shown in FIG. 1 and an equivalent circuit diagram showing a modified structure example of a pixel part;

FIG. 18 is a diagram showing a fifth modified example of the endoscope device shown in FIG. 1 and an equivalent circuit diagram showing a modified structure example of a pixel part;

FIG. 19 is a timing chart for explaining an operation of the fifth modified example of the endoscope device shown in FIG. 1;

FIG. 20 is a schematic diagram for explaining an electric charge storing operation of the fifth modified example of the endoscope device shown in FIG. 1;

FIGS. 21A and 21B are schematic plan views showing a schematic structure of another example of a solid-state image pick-up element for explaining the one exemplary embodiment of the present invention;

FIG. 22 is a diagram showing an equivalent circuit of a pixel part in the solid-state image pick-up element shown in FIG. 21;

FIG. 23 is a schematic plan view showing a layout example on a plane of the pixel part of the solid-state image pick-up element shown in FIG. 21;

FIG. 24 is a schematic sectional view taken along a line A-A′ of the pixel part shown in FIG. 23;

FIG. 25 is a schematic sectional view taken along a line B-B′ of the pixel part shown in FIG. 23; and

FIG. 26 is a diagram showing a modified example of the solid-state image pick-up element shown in FIG. 21.

### DETAILED DESCRIPTION

Now, an exemplary embodiment of the present invention will be described below by referring to the drawings.

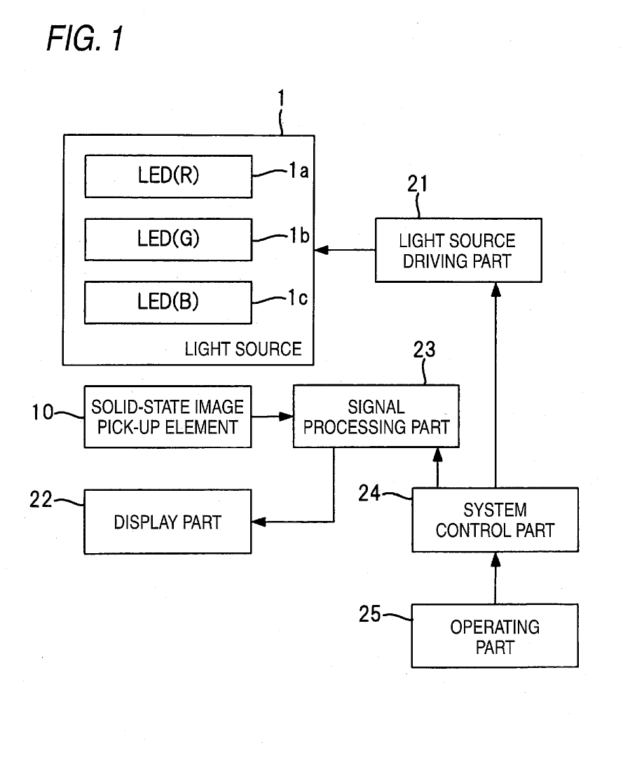

FIG. 1 is a diagram showing a schematic structure of an endoscope device that explains one exemplary embodiment of the present invention. An endoscope device shown in FIG. 1 includes a light source 1, a solid-state image pick-up element 10, a light source driving part 21, a signal processing part 23, a system control part 24, a display part 22 and an operating part 25.

The light source 1 includes an LED 1a for emitting lights of a wavelength area of R (ordinarily, about 550 nm to about 700 nm), an LED 1b of a wavelength area of G (ordinarily, about 450 nm to about 610 nm) and an LED 1c for emitting lights of a wavelength area of B (ordinarily, about 380 nm to about 520 nm). The LEDs are exemplified as examples, however, any light source that may emit the lights of the above-described wavelength areas of R, G and B may be employed.

The LED 1a, 1b and 1c are respectively independently driven by the light source driving part 21. The lights emitted from the LEDs respectively are applied to an object to be shot or recorded in the front part of the solid-state image pick-up element 10 through a light guide not shown in the drawing.

The signal processing part 23 applies a signal process to an image pick-up signal outputted from the solid-state image pick-up element 10 to generate image data. The generated image data is recorded on a recording medium or displayed on the display part 22.

The system control part 24 generally controls an entire part of the endoscope device. The operating part 25 is an interface for carrying out various kinds of operations of the endoscope device.

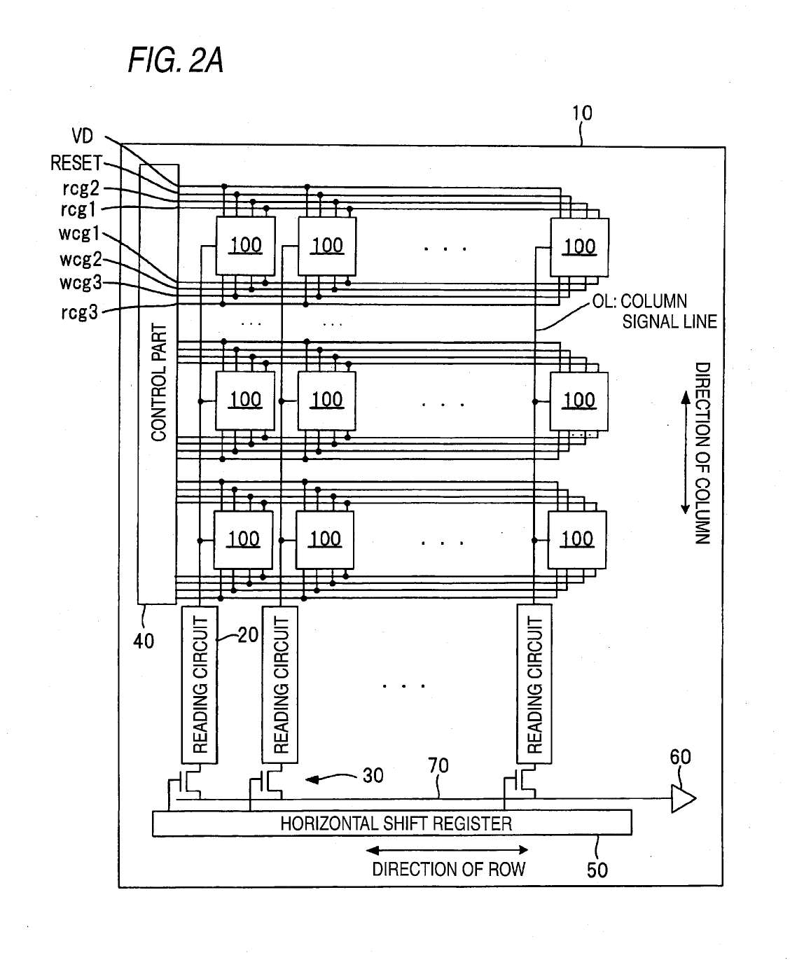

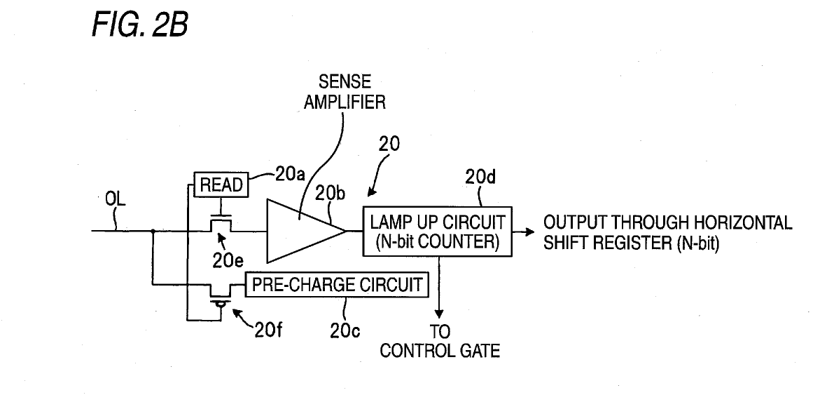

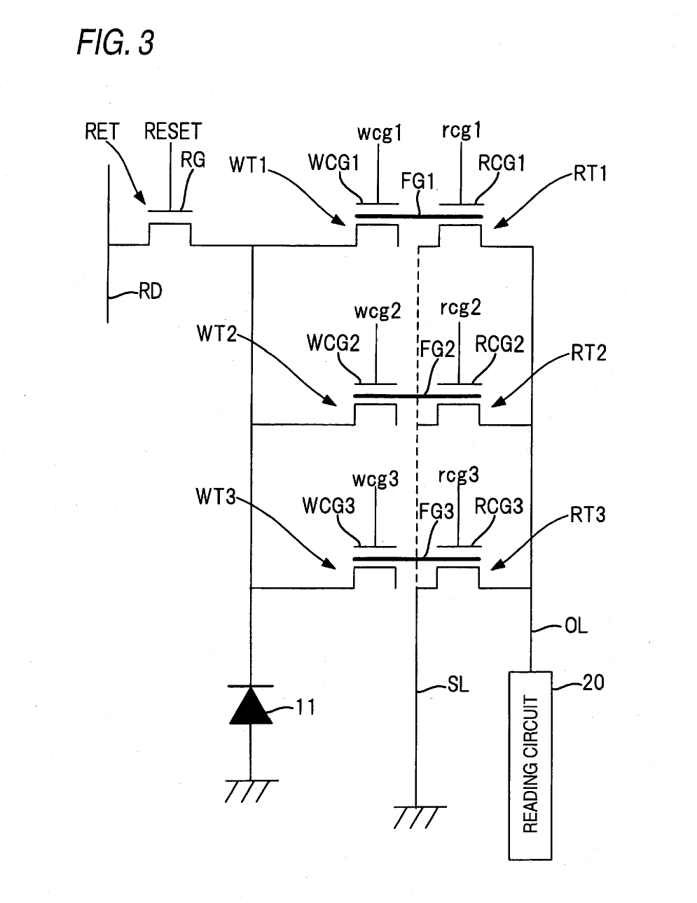

FIG. 2A is a diagram showing a schematic structure of the solid-state image pick-up element 10 in FIG. 1. FIG. 3 is a diagram showing an equivalent circuit of an inner structure of one pixel part 100 showing in FIG. 2A.

The solid-state image pick-up element 10 includes a plurality of pixel parts 100 arranged in the form of an array (here, in a square grid form) in the directions of rows and the directions of columns orthogonal thereto on the same plane.

The pixel part 100 includes an N type silicon substrate and an N type impurity layer 11 formed in a semiconductor substrate composed of a P well layer formed thereon. The N type impurity layer 11 is formed in the P well layer to form a photodiode (PD) functioning as a photoelectric conversion part by a PN junction of the N type impurity layer 11 and the P well layer. The N type impurity layer 11 is referred to as a photoelectric conversion part 11 hereinafter.

The pixel part 100 includes three electric charge storage parts as a plurality of electric charge storage parts that may selectively store electric charges generated in the photoelectric conversion part 11. The three electric charge storage parts are referred to as a first electric charge storage part, a second electric charge storage part and a third electric charge storage part hereinafter.

The first electric charge storage part includes a writing transistor WT1 and a reading transistor RT1.

The writing transistor WT1 includes a floating gate FG1 as an electric charge storage area that electrically floats, and is an MOS transistor of a two-terminal structure having the photoelectric conversion part 11 as a source and a drain (a two-terminal structure having a source connected to the photoelectric conversion part 11 and a writing control gate WCG1) and an operation of the writing transistor WT1 is controlled by the writing control gate WCG1. The writing control gate WCG1 is connected to a control part 40 through a writing control line wcg1. In the writing transistor WT1, when a writing pulse is applied to the writing control gate WCG1, the electric charge generated in the photoelectric conversion part 11 is injected to and stored in the floating gate FG1 by an FN tunnel injection for injecting the electric charge by using a Fowler-Nordheim (F-N) tunnel current, a direct tunnel injection, a hot electron injection or the like. The writing transistor WT1 may be a three-terminal structure that has the photoelectric conversion part 11 as a source and a drain separately from the source.

The reading transistor RT1 has the floating gate FG1 common to the writing transistor WT1 and is an MOS transistor having a drain connected to a column signal line OL and a source commonly connected to sources of below-described reading transistors RT2 and RT3 and an operation of the reading transistor RT1 is controlled by a reading control gate RCG1. The reading control gate RCG1 is connected to the control part 40 through a reading control line rcg1. In the reading transistor RT1, since a threshold voltage changes correspondingly to an amount of electric charge stored in the floating gate FG1, the change of the threshold voltage (a variation obtained when the threshold voltage is set as a reference under a state that the electric charge is not stored in the floating gate FG1) may be read as an image pick-up signal corresponding to the electric charge stored in the floating gate FG1.

The floating gate FG1 is not limited to a structure common to the writing transistor WT1 and the reading transistor RT1, and floating gates may be respectively separately provided in the writing transistor WT1 and the reading transistor RT1 and the two separate floating gates FG1 may be electrically connected together by a wiring.

The second electric charge storage part includes a writing transistor WT2 and a reading transistor RT2.

The writing transistor WT2 includes a floating gate FG2 as an electric charge storage area that electrically floats, and is an MOS transistor of a two-terminal structure having the photoelectric conversion part 11 as a source and a drain (a two-terminal structure having a source connected to the photoelectric conversion part 11 and a writing control gate WCG2) and an operation of the writing transistor WT2 is controlled by the writing control gate WCG2. The writing control gate WCG2 is connected to the control part 40 through a writing control line wcg2. In the writing transistor WT2, when a writing pulse is applied to the writing control gate WCG2, the electric charge generated in the photoelectric conversion part 11 is injected to and stored in the floating gate FG2 by an FN tunnel injection, a direct tunnel injection, a hot electron injection or the like. The writing transistor WT2 may be a three-terminal structure that has the photoelectric conversion part 11 as a source and a drain separately from the source. In this case, the drains of the writing transistor WT1 and the writing transistor WT2 may be made to be common.

The reading transistor RT2 has the floating gate FG2 common to the writing transistor WT2 and is an MOS transistor having a drain connected to the column signal line OL and a source commonly connected to sources of the reading transistors RT1 and RT3 and an operation of the reading transistor RT2 is controlled by a reading control gate RCG2. The reading control gate RCG2 is connected to the control part 40 through a reading control line rcg2. In the reading transistor RT2, since a threshold voltage changes correspondingly to an amount of electric charge stored in the floating gate FG2, the change of the threshold voltage (a variation obtained when the threshold voltage is set as a reference under a state that the electric charge is not stored in the floating gate FG2) may be read as an image pick-up signal corresponding to the electric charge stored in the floating gate FG2.

The floating gate FG is not limited to a structure common to the writing transistor WT2 and the reading transistor RT2, and floating gates may be respectively separately provided in the writing transistor WT2 and the reading transistor RT2 and the two separate floating gates FG2 may be electrically connected together by a wiring.

The third electric charge storage part includes a writing transistor WT3 and a reading transistor RT3.

The writing transistor WT3 includes a floating gate FG3 as an electric charge storage area that electrically floats, and is an MOS transistor of a two-terminal structure having the photoelectric conversion part 11 as a source and a drain (a two-terminal structure having a source connected to the photoelectric conversion part 11 and a writing control gate WCG3) and an operation of the writing transistor WT3 is controlled by the writing control gate WCG3. The writing control gate WCG3 is connected to the control part 40 through a writing control line wcg3. In the writing transistor WT3, when a writing pulse is applied to the writing control gate WCG3, the electric charge generated in the photoelectric conversion part 11 is injected to and stored in the floating gate FG3 by an FN tunnel injection, a direct tunnel injection, a hot electron injection or the like. The writing transistor WT3 may be a three-terminal structure that has the photoelectric conversion part 11 as a source and a drain separately from the source. In this case, the drains of the writing transistor WT1, the writing transistor WT2 and the writing transistor WT3 may be made to be common.

The reading transistor RT3 has the floating gate FG3 common to the writing transistor WT31 and is an MOS transistor having a drain connected to the column signal line OL and a source commonly connected to the sources of the reading transistors RT1 and RT2 and an operation of the reading transistor RT3 is controlled by a reading control gate RCG3. The reading control gate RCG3 is connected to the control part 40 through a reading control line rcg3. In the reading transistor RT3, since a threshold voltage changes correspondingly to an amount of electric charge stored in the floating gate FG3, the change of the threshold voltage (a variation obtained when the threshold voltage is set as a reference under a state that the electric charge is not stored in the floating gate FG3) may be read as an image pick-up signal corresponding to the electric charge stored in the floating gate FG3.

The floating gate FG3 is not limited to a structure common to the writing transistor WT3 and the reading transistor RT3, and floating gates may be respectively separately provided in the writing transistor WT3 and the reading transistor RT3 and the two separate floating gates FG3 may be electrically connected together by a wiring.

The sources of the reading transistor RT1, the reading transistor RT2 and the reading transistor RT3 are respectively connected to a ground potential through a source line SL.

The pixel part 100 further includes a reset transistor RET for discharging the electric charges stored in the photoelectric conversion part 11. A reset gate RG of the reset transistor RET is connected to the control part 40 through a reset line RESET. When a reset pulse is applied through the reset line RESET from the control part 40, the reset transistor RET is turned on to discharge the electric charge stored in the photoelectric conversion part 11 to a drain of the reset transistor RET. The drain of the reset transistor RET is connected to a reset power line VD through a reset drain line RD.

The solid-state image pick-up element 10 includes the control part 40 for driving and controlling the pixel parts 100 respectively, a reading circuit 20 for detecting the threshold voltages of the reading transistor RT1, the reading transistor RT2 and the reading transistor RT3 respectively, a horizontal shift register 50 and a horizontal selecting transistor 30 that control the threshold voltages of one line detected in the reading circuit 20 to be sequentially read as the image pick-up signals to a signal line 70 and an output amplifier 60 connected to the signal line 70.

The reading circuit 20 is provided correspondingly to each column including a plurality of pixel parts 100 arranged in the direction of the column and connected to the drains respectively of the reading transistors RT1, RT2 and RT3 of the pixel parts 100 of the corresponding column through the column signal line OL. The reading circuit 20 is also connected to the control part 40.

As shown in FIG. 2B, the reading circuit 20 includes a reading control part 20a, a sense amplifier 20b, a pre-charge circuit 20c, a lamp up circuit 20d and transistors 20e and 20f.

When the reading control part 20a reads the signal from the first (the second, the third) electric charge storage part of the pixel part 100, the reading control part 20a turns on the transistor 20f to supply a drain voltage (pre-charge) to the drain of the reading transistor RT1 (RT2, RT3) of the pixel part 100 through the column signal line OL from the pre-charge circuit 20c. Then, the reading control part 20a turns on the transistor 20e to electrically conduct the drain of the reading transistor RT1 (RT2, RT3) to the sense amplifier 20b.

The sense amplifier 20b monitors the voltage of the drain of the reading transistor RT1 (RT2, RT3) of the pixel part 100 to detect that the voltage changes and informs the lamp up circuit 20d that the voltage changes. For instance, the sense amplifier 20b detects that the drain voltage pre-charged by the pre-charge circuit 20c drops to invert an output of the sense amplifier.

The lamp up circuit 20d incorporates an N-bit counter (for instance, N=about 8 to 12) to supply a lamp wave form voltage that gradually increases or gradually decreases to the reading control gate RCG1 (RCG2, RCG3) of the reading transistor RT1 (RT2, RT3) of the pixel part 100 through the control part 40 and output a count value (N combinations of 1 and 0) corresponding to the value of the lamp wave form voltage.

When the voltage of the reading control gate RCG1 (RCG2, RCG3) exceeds the threshold voltage of the reading transistor RT1 (RT2, RT3), the reading transistor RT1 (RT2, RT3) is electrically conducted. At this time, the pre-charged potential of the column signal line OL drops. This drop is detected by the sense amplifier 20b and an inversion signal is outputted. The lamp up circuit 20d holds (latches) the count value corresponding to the value of the lamp wave form voltage when the lamp up circuit 20d receives the inversion signal. Thus, the change (the image pick-up signal) of the threshold voltage may be read as a digital value (a combination of 1 and 0).

When one horizontal selecting transistor 30 is selected by the horizontal shift register 50, the counter value held by the lamp up circuit 20d connected to the horizontal selecting transistor 30 is outputted to the signal line 70 and outputted from the output amplifier 60 as the image pick-up signal.

A method for reading the change of the threshold voltage of the reading transistor RT1 (RT2, RT3) by the reading circuit 20 is not limited to the above-described method. For instance, a drain current of the reading transistor RT1 (RT2, RT3) obtained when a prescribed voltage is applied to the reading control gate RCG1 (RCG2, RCG3) and the drain of the reading transistor RT1 (RT2, RT3) may be read as the image pick-up signal.

The control part 40 independently controls the writing transistors WT1, WT2 and WT3 to be driven so as to inject and store the electric charges generated in the photoelectric conversion part 11 in the floating gates FG1, FG2 and FG3. As a method for injecting the electric charges to the floating gates FG1, FG2 and FG3, the FN tunnel injection, the direct tunnel injection, the hot electron injection or the like are exemplified.

Further, the control part 40 controls the reading circuit 20 to be driven so as to independently read the image pick-up signals corresponding to the electric charges stored in the floating gates FG1, FG2 and FG3.

Further, the control part 40 drives the electric charges stored in the floating gates FG1, FG2 and FG3 to be discharged to an external part and erased. For instance, a positive voltage is applied to the semiconductor substrate to apply a negative voltage to the writing control gate WCG1 and the reading control gate RCG1 respectively. Thus, the electric charge stored in the floating gate FG1 is drawn out to the semiconductor substrate to erase the electric charge. When the electric charge stored in the floating gate FG2 is erased, the positive voltage is applied to the semiconductor substrate to apply the negative voltage to the writing control gate WCG2 and the reading control gate RCG2 respectively. When the electric charge stored in the floating gate FG3 is erased, the positive voltage is applied to the semiconductor substrate to apply the negative voltage to the writing control gate WCG3 and the reading control gate RCG3 respectively.

A color filter is not provided in an upper part of the photoelectric conversion part 11 of each pixel part 100. All lights incident on the solid-state image pick-up element 10 are incident on each photoelectric conversion part 11.

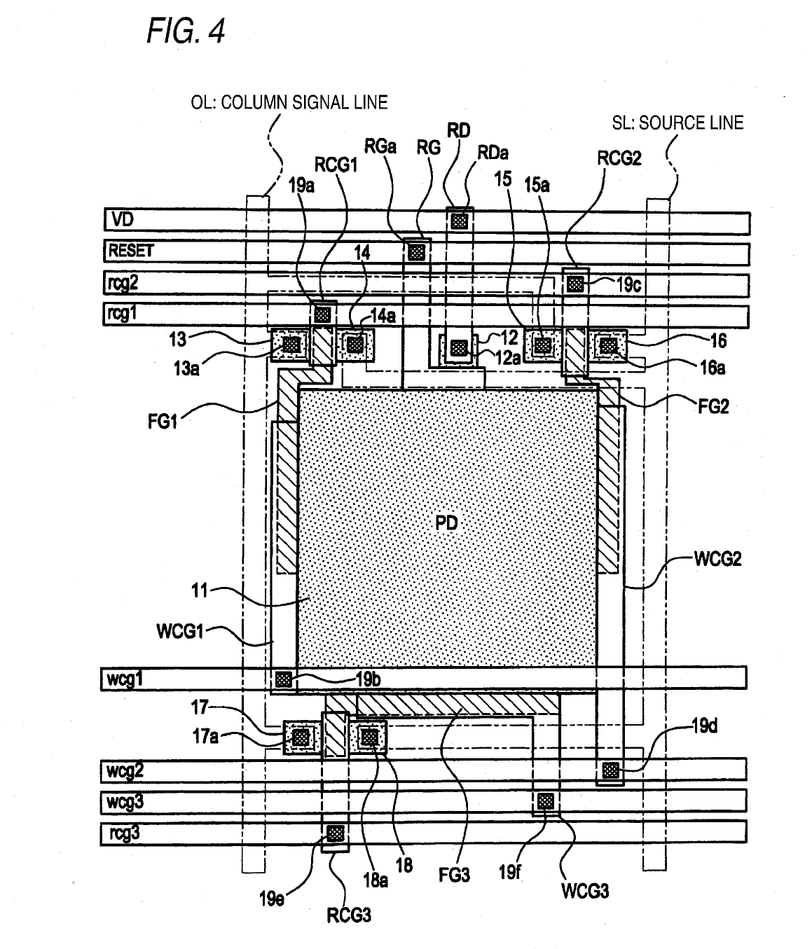

FIG. 4 is a schematic plan view showing a layout example of the pixel part 100 based on the equivalent circuit diagram shown in FIG. 3.

In the P well layer of the pixel part 100, the photoelectric conversion part 11 is formed. On an upper part of the photoelectric conversion part 11, a drain 13 of the reading transistor RT1, a source 14 of the reading transistor RT1, a drain 12 of the reset transistor RET, a drain 15 of the reading transistor RT2 and a source 16 of the reading transistor RT2 are formed and arranged in the directions of columns a little separated from the photoelectric conversion part 11. Further, in a lower part of the photoelectric conversion part 11, a drain 17 of the reading transistor RT3 and a source 18 of the reading transistor RT3 are formed and arranged in the directions of columns a little separated from the photoelectric conversion part 11.

On the P well layer, an oxide film that is not shown in the drawing is formed and the floating gate FG1, the floating gate FG2 and the floating gate FG3 are formed thereon. The floating gate FG1 extends along a left side to an upper side of the photoelectric conversion part 11 and to an upper part between the drain 13 and the source 14. The floating gate FG2 is formed so as to extend along a right side to an upper side of the photoelectric conversion part 11 and to an upper part between the drain 15 and the source 16. The floating gate FG3 is formed so as to extend along a lower side of the photoelectric conversion part 11 and to an upper part between the drain 17 and the source 18.

On the floating gates FG1, FG2 and FG3, insulating films are provided. On upper layers thereof, the writing control gates WCG1, WCG2 and WCG3, the reading control gates RCG1, RCG2 and RCG3, the reset gate RG and the reset drain line RD are formed.

The writing control gate WCG1 is formed so as to be overlapped on the floating gate FG1. The reading control gate RCG1 is formed so as to be overlapped on the floating gate FG1 in the upper part between the drain 13 and the source 14.

The writing control gate WCG2 is formed so as to be overlapped on the floating gate FG2. The reading control gate RCG2 is formed so as to be overlapped on the floating gate FG2 in the upper part between the drain 15 and the source 16.

The writing control gate WCG3 is formed so as to be overlapped on the floating gate FG3. The reading control gate RCG3 is formed so as to be overlapped on the floating gate FG3 in the upper part between the drain 17 and the source 18.

The rest gate RG is formed in an upper part between the photoelectric conversion part 11 and the drain 12. The reset drain line RD is formed so as to extend from an upper part of the drain 12 to a lower part of the reset power line VD and is electrically connected to the drain 12 through a contact part 12a in the upper part of the drain 12, and is electrically connected to the reset power line VD through a contact part RDa in the lower part of the reset power line VD.

On upper layers of the writing control gates WCG1, WCG2 and WCG3, the reading control gates RCG1, RCG2 and RCG3, the reset gate RG, and the reset drain line RD, a global wiring (the reset power line VD, the reset line RESET, the reading control line rcg2, the reading control line rcg1, the writing control line wcg1, the writing control line wcg2, the writing control line wcg3 and the reading control liner rcg3) is formed which extends in the directions of rows through insulating films.

The reading control gate RCG1 extends to a lower part of the reading control line rcg1, and is electrically connected to the reading control line rcg1 herein through a contact part 19a. The writing control gate WCG1 extends to a lower part of the writing control line wcg1, and is electrically connected to the writing control line wcg1 herein through a contact part 19b.

The reading control gate RCG2 extends to a lower part of the reading control line rcg2, and is electrically connected to the reading control line rcg2 herein through a contact part 19c. The writing control gate WCG2 extends to a lower part of the writing control line wcg2, and is electrically connected to the writing control line wcg2 herein through a contact part 19d.

The reading control gate RCG3 extends to a lower part of the reading control line rcg3, and is electrically connected to the reading control line rcg3 herein through a contact part 19e. The writing control gate WCG3 extends to a lower part of the writing control line wcg3, and is electrically connected to the writing control line wcg3 herein through a contact part 19f.

The reset gate RG extends to a lower part of the reset line RESET and is electrically connected to the reset line RESET herein through a contact part RGa.

On the global wiring extending in the directions of the rows, an insulating film is formed, and, on an upper layer thereof, a global wiring (the column signal line OL, the source line SL) is formed that extends in the directions of columns.

The column signal line OL extends to the upper parts of the drain 13, the drain 15 and the drain 17 respectively and is electrically connected to the drain 13, the drain 15 and the drain 17 herein through contact parts 13a, 15a and 17a.

The source line SL extends to the upper parts of the source 14, the source 16 and the source 18 respectively, and is electrically connected to the source 14, the source 16 and the source 18 herein through contact parts 14a, 16a and 18a.

In the layout example in FIG. 4, the drains of the writing transistors WT1, WT2 and WT3 are omitted. The writing transistors WT1, WT2 and WT3 are respectively formed as the MOS transistors having the two-terminal structures having sources (commonly used as drains) connected to the photoelectric conversion part 11. A two-terminal device includes a resistance, a coil, a capacitor, a diode or the like, and does not include an active device for switching or signal amplification.

It is to be understood as a common sense that a transistor as the active device for selecting a pixel, resetting, recording and reading a signal in an ordinary solid-state image pick-up element does not function in two terminals, and nobody tries to use the transistor having the two-terminals.

In the structure of the pixel part 100 shown in FIG. 3, since the floating gate FG1 is shared by the writing transistor WT1 and the reading transistor RT1, the writing transistor WT1 is exclusively required to carry out a single operation of a writing operation (an injection of the electric charge to and a recording in the floating gate FG1) and a movement of the electric charge only in one direction. When the signal is read, the signal may be read in the adjacent reading transistor RT1 side by the above-described shared FG structure. Thus, it is recognized that the writing transistor WT1 having the two-terminal structure has no problem in operation. The above-described matter may be applied to the writing transistors WT2 and WT3.

In the case of the solid-state image pick-up element 10, since the three electric charge storage parts need to be formed in the pixel part 100, a degree of freedom in design is deteriorated. Thus, the writing transistors WT1, WT2 and WT3 have the two-terminal structures, the structure is effectively simplified. In accordance with such a structure, the size of the pixel part 100 or the size of a chip may be reduced so that the formation of multi-pixels or a miniaturization may be realized.

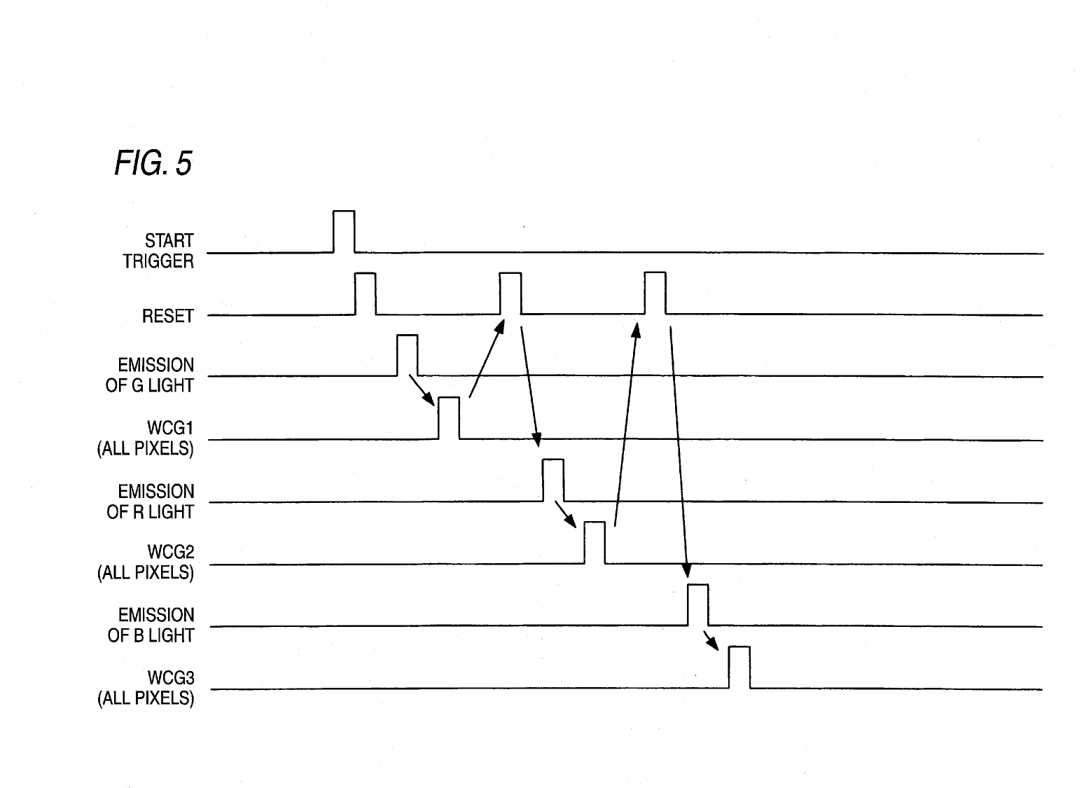

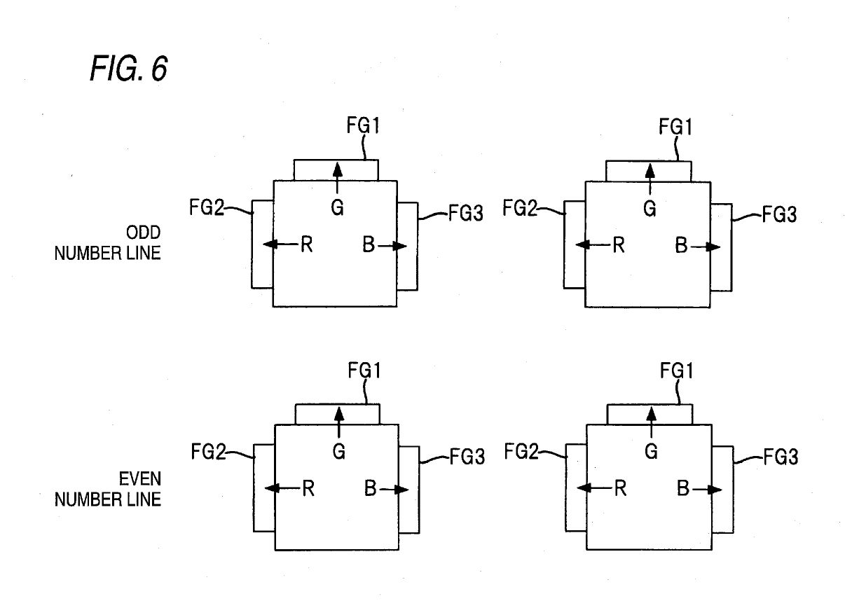

An operation of the endoscope device constructed as described above will be described below. FIG. 5 is a timing chart for explaining the operation of the endoscope device shown in FIG. 1. FIG. 6 is a schematic view for explaining the operation of the endoscope device shown in FIG. 1. In FIG. 6, four pixel parts of 2 rows x 2 columns in total are schematically shown.

When the operating part 25 is operated to instruct an object to be shot or recorded, this instruction is inputted to the system control part 24 and the system control part 24 informs the solid-state image pick-up element 10 of the instruction for shooting or recording the object.

When the solid-state image pick-up element 10 receives the instruction for shooting or recording the object, the control part 40 considers it as a start trigger to supply a reset pulse to the reset gates RG of the reset transistors RET of all the pixel parts 100. Thus, unnecessary electric charges respectively stored in the photoelectric conversion parts 11 of the pixel parts 100 are discharged to the drains of the reset transistors RET.

After the reset operation is completed, the system control part 24 outputs an instruction to the light source driving part 21 to emit the G light from the LED 1b. In FIG. 5, the G light is emitted after a little time when the reset pulse is supplied, however, the G light may be emitted at the same time as the completion of the reset operation.

The G light is emitted, for instance, only during an exposure period set by the endoscope device. During the emission of the G light, in the pixel parts 100 of the solid-state image pick-up element 10 respectively, lights incident from the object are incident on the photoelectric conversion parts 11 and the electric charges corresponding to the G light are generated and stored.

After the exposure period is finished, the control part 40 supplies writing pulses to the writing control gates WCG1 of all the pixel parts 100 to store the electric charges generated in the photoelectric conversion parts 11 during the exposure period in the floating gates FG1. In supplying the writing pulse, either a method for starting the supply of the writing pulse at the same time as the completion of the exposure period or a method for starting the supply of the writing pulse at the same time as the start of the exposure period and completing the supply of the writing pulse at the same time as the completion of the exposure period may be employed.

In accordance with the supply of the writing pulses, as shown in FIG. 6, the electric charges (the electric charges by the G light, shown by “G” in the drawing) generated in the pixel parts 100 are stored respectively in the floating gates FG1 of the pixel parts 100.

When the storage of the electric charges in the floating gates FG1 is finished, the control part 40 supplies again reset pulses to the reset gates RG of the reset transistors RET of all the pixel parts 100. Thus, remaining electric charges which are hardly injected to the floating gates FG1 from the photoelectric conversion parts 11 to be left are discharged to the drains of the reset transistors RET.

After the second reset operation is completed, the system control part 24 outputs an instruction to the light source driving part 21 to emit the R light from the LED 1a. In FIG. 5, the R light is emitted after a little time when the reset pulse is supplied, however, the R light may emitted at the same time as the completion of the reset operation.

The R light is emitted, for instance, only during an exposure period set by the endoscope device. During the emission of the R light, in the pixel parts 100 of the solid-state image pick-up element 10 respectively, lights incident from the object are incident on the photoelectric conversion parts 11 and the electric charges corresponding to the R light are generated and stored.

After the exposure period is finished, the control part 40 supplies writing pulses to the writing control gates WCG2 of all the pixel parts 100 to store the electric charges generated in the photoelectric conversion parts 11 during the exposure period in the floating gates FG2. In supplying the writing pulse, either a method for starting the supply of the writing pulse at the same time as the completion of the exposure period or a method for starting the supply of the writing pulse at the same time as the start of the exposure period and completing the supply of the writing pulse at the same time as the completion of the exposure period may be employed.

In accordance with the supply of the writing pulses, as shown in FIG. 6, the electric charges (the electric charges by the R light, shown by “R” in the drawing) generated in the pixel parts 100 are stored respectively in the floating gates FG2 of the pixel parts 100.

When the storage of the electric charges in the floating gates FG2 is finished, the control part 40 supplies again reset pulses to the reset gates RG of the reset transistors RET of all the pixel parts 100. Thus, remaining electric charges which are hardly injected to the floating gates FG2 from the photoelectric conversion parts 11 to be left are discharged to the drains of the reset transistors RET.

After the third reset operation is completed, the system control part 24 outputs an instruction to the light source driving part 21 to emit the B light from the LED 1c. In FIG. 5, the B light is emitted after a little time when the reset pulse is supplied, however, the light may be emitted at the same time as the completion of the reset operation.

The B light is emitted, for instance, only during an exposure period set by the endoscope device. During the emission of the B light, in the pixel parts 100 of the solid-state image pick-up element 10 respectively, lights incident from the object are incident on the photoelectric conversion parts 11 and the electric charges corresponding to the B light are generated and stored.

After the exposure period is finished, the control part 40 supplies writing pulses to the writing control gates WCG3 of all the pixel parts 100 to store the electric charges generated in the photoelectric conversion parts 11 during the exposure period in the floating gates FG3. In supplying the writing pulse, either a method for starting the supply of the writing pulse at the same time as the completion of the exposure period or a method for starting the supply of the writing pulse at the same time as the start of the exposure period and completing the supply of the writing pulse at the same time as the completion of the exposure period may be employed.

In accordance with the supply of the writing pulses, as shown in FIG. 6, the electric charges (the electric charges by the B light, shown by “B” in the drawing) generated in the pixel parts 100 are stored respectively in the floating gates FG3 of the pixel parts 100.

In the solid-state image pick-up element 10, since the writing control gate WCG1, the writing control gate WCG2 and the writing control gate WCG3 are respectively connected to the different control lines (wcg1, wcg2, wcg3), as described above, the electric charges generated in the photoelectric conversion parts 11 by the exposure operations of three times respectively may be selectively stored in the respectively different floating gates.

After the electric charges are completely stored in the floating gates FG3, the control part 40 pre-charges the drains of the reading transistors RT1 of the pixel parts 100 respectively in a first line to begin to apply the lamp wave form voltage to the reading control gates RCG1 of the pixel parts 100 of the first line (count values after the start of application of the lamp wave form voltage are up counted from, for instance, an initial value (for instance, zero). Then, the count values corresponding to the value of the lamp wave form voltage when the drain potentials of the reading transistors RT1 of the first line drop are respectively held in the reading circuits 20 and the count values are outputted from the output amplifier 60 as the image pick-up signals. The control part 40 carries out a similar driving operation in lines after a second line to output first image pick-up signals (G signals) corresponding to the electric charges stored in the floating gates FG1 of all lines.

Then, the control part 40 pre-charges the drains of the reading transistors RT2 of the pixel parts 100 respectively in the first line to begin to apply the lamp wave form voltage to the reading control gates RCG2 of the pixel parts 100 of the first line (count values after the start of application of the lamp wave form voltage are up counted from, for instance, an initial value (for instance, zero). Then, the count values corresponding to the value of the lamp wave form voltage when the drain potentials of the reading transistors RT2 of the first line drop are respectively held in the reading circuits 20 and the count values are outputted from the output amplifier 60 as the image pick-up signals. The control part 40 carries out a similar driving operation in lines after a second line to output second image pick-up signals (R signals) corresponding to the electric charges stored in the floating gates FG2 of all lines.

Then, the control part 40 pre-charges the drains of the reading transistors RT3 of the pixel parts 100 respectively in the first line to begin to apply the lamp wave form voltage to the reading control gates RCG3 of the pixel parts 100 of the first line (count values after the start of application of the lamp wave form voltage are up counted from, for instance, an initial value (for instance, zero). Then, the count values corresponding to the value of the lamp wave form voltage when the drain potentials of the reading transistors RT3 of the first line drop are respectively held in the reading circuits 20 and the count values are outputted from the output amplifier 60 as the image pick-up signals. The control part 40 carries out a similar driving operation in lines after a second line to output third image pick-up signals (B signals) corresponding to the electric charges stored in the floating gates FG3 of all lines.

After the third image pick-up signals are outputted, the control part 40 sets the potentials of the writing control gates WCG1, WCG2 and WCG3 and the reading control gates RCG1, RCG2 and RCG3 of all the pixel parts 100 to—Vcc and the potential of the semiconductor substrate to Vcc. Thus, the electric charges stored in the floating gates FG1, FG2 and FG3 are drawn out to the semiconductor substrate and erased.

The above-described operations are carried out within one frame period.

In accordance with the above-described operations, the G signals, the R signals and the B signals are obtained respectively from the pixel parts 100 of the solid-state image pick-up element 10. Accordingly, a YC signal is formed from these signals without carrying out a signal interpolating process so that color image data of a JPEG form may be formed. By a color image based on the color image data, the same state as that the object is observed by the naked eye may be reproduced on the display part 22.

As described above, according to the endoscope device shown in FIG. 1, every time an exposure process is carried out, the signals corresponding to the electric charges obtained by the exposure process do not need to be read, and the signals may be read together after the exposure processes of the three times. As a result, since intervals between the exposure processes of the three times may be shorted, a color divergence arising when the object to be shot or recorded moves may be suppressed. Accordingly, a diagnostic accuracy during an inspection by the endoscope device may be improved.

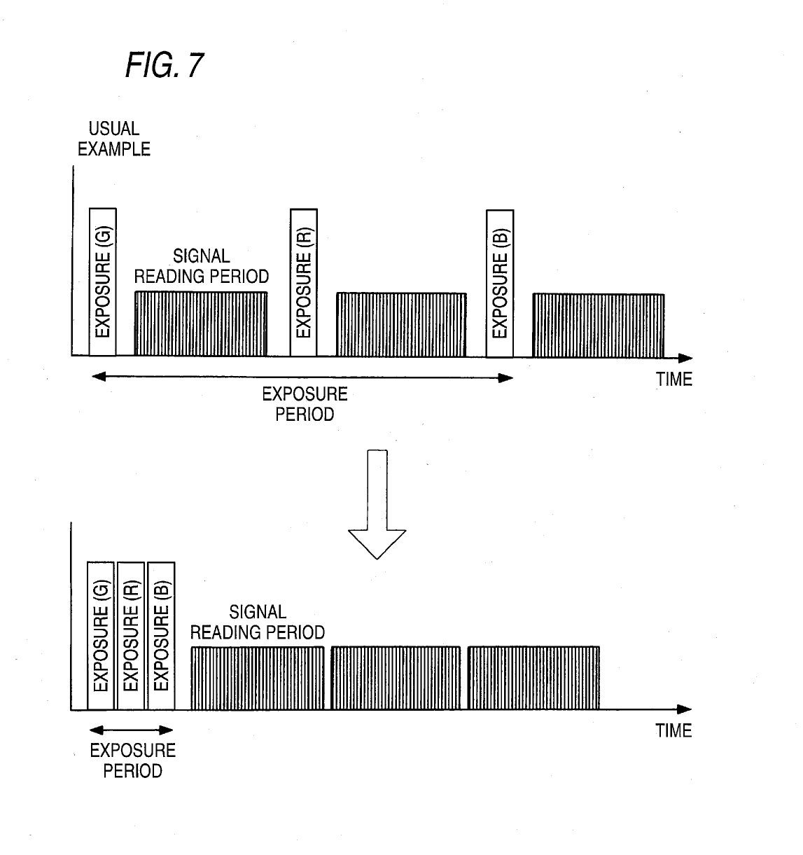

Further, the endoscope device shown in FIG. 1 carries out shooting or recording operations during the one frame period as a period for obtaining the image data of one frame and reads the image pick-up signals after the shooting or recording operations of the three times. When the shooting operations of the three times are tried to be carried out during the one frame period, the intervals of the shooting operations of the three times need to be shortened. As shown in an upper stage of FIG. 7, in an ordinary solid-state image pick-up element, every time the shooting or recording operation is finished, the image pick-up signals need to be read. In order to shorten the intervals between the shooting operations, the image pick-up signals need to be read at high speed. When the image pick-up signals are read at higher speed, the heat generation of the element is the more increased. In the endoscope device, the heat generation of an end part inserted into the body needs to be suppressed as much as possible. When the heat generation is increased, a cooling mechanism is necessary in the end part. Thus, the miniaturization of the end part is prevented. According to the endoscope device shown in FIG. 1, as shown in a lower stage of FIG. 7, even when the image pick-up signals are not read at high speed, the intervals between the shooting or recording operations may be shortened. Accordingly, the heat generation in the end part may be suppressed and the miniaturization of the end part may be realized.

Further, according to the endoscope device shown in FIG. 1, before the electric charges are stored in the floating gates FG2 and FG, since the electric charges in the photoelectric conversion parts 11 are driven to be temporarily discharged to the rest drains, the electric charges during the exposure processes by the different lights may be prevented from being mixed together to prevent a color mixture and more improve an image quality.

In the above explanation, the first electric charge storage part, the second electric charge storage part and the third electric charge storage part are respectively formed with the two MOS transistors including the writing transistors WT and the reading transistors RT, however, the electric charge storage parts may be respectively formed with one transistors.

For instance, in FIG. 3, the reading transistors RT1, RT2 and RT3 may be omitted and the writing transistors WT1, WT2 and WT3 may have drains to which the reading circuit 20 is connected through the column signal line OL. In this structure, the G signal may be read by pre-charging the drain of the writing transistor WT1 to apply the lamp wave form voltage to the writing control gate WCG1. The R signal may be read by pre-charging the drain of the writing transistor WT2 to apply the lamp wave form voltage to the writing control gate WCG2. The B signal may be read by pre-charging the drain of the writing transistor WT3 to apply the lamp wave form voltage to the writing control gate WCG3.

As described above, when the electric charge storage part is realized by one transistor, the transistor may employ other structure than a MOS structure. For instance, may be employed an MNOS type transistor structure in which a floating gate FG1 is made of a nitride film and a writing control gate WCG1 is directly formed on the nitride film or an MONOS type transistor structure in which a floating gate FG1 is made of a nitride film. In both the cases, the nitride film (N) functions as an electric charge storage area for storing the electric charges.

Further, in the above explanation, as the light source, the light source is used that emits the lights of primary colors, however, a light source may be employed that emits three lights of complementary colors (cyan, magenta, yellow) to similarly form the color image data.

Now, modified examples of the endoscope device shown in FIG. 1 will be described below.

### First Modified Example

An endoscope device of a first modified example has the same structure as that of the endoscope device shown in FIG. 1 and only an operation thereof is different from the endoscope device shown in FIG. 1. Now, the operation will be described below.

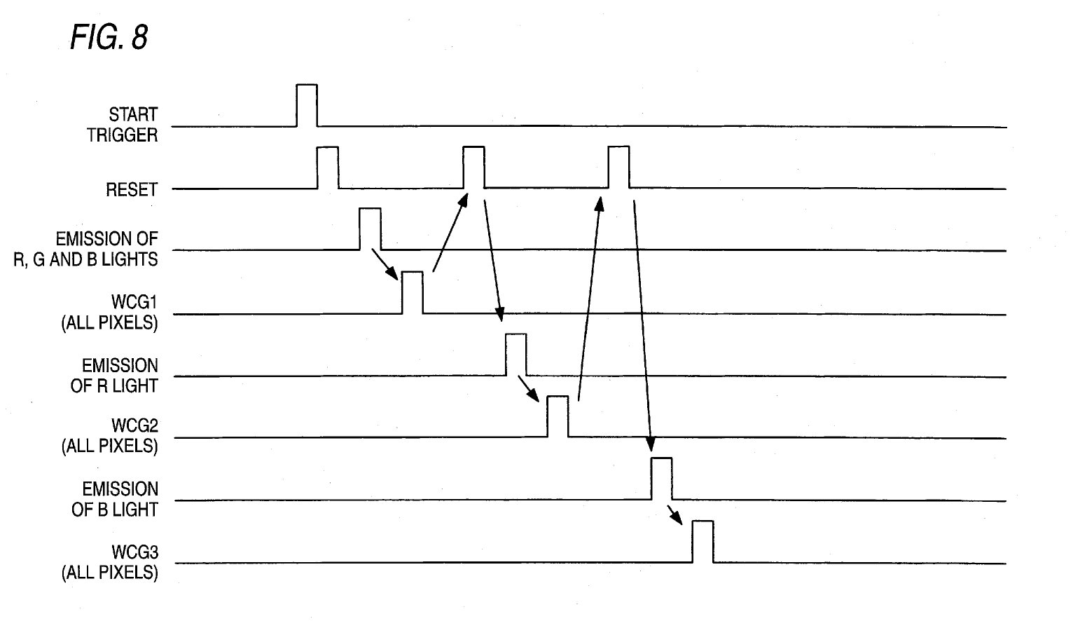

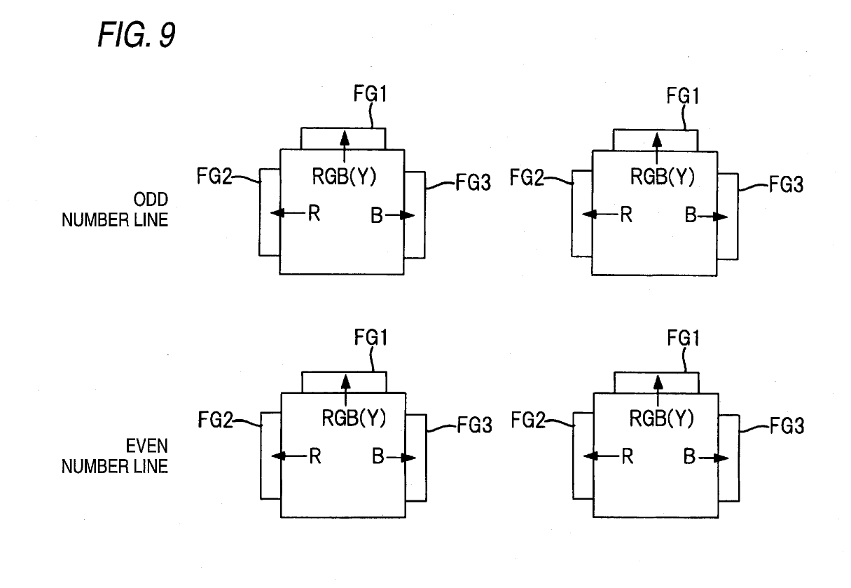

FIG. 8 is a timing chart for explaining an operation of the first modified example. FIG. 9 is a schematic diagram for explaining an operation of the first modified example. In FIG. 9, four pixel parts of 2 rows×2 columns in total are schematically shown.

When an operating part 25 is operated to instruct an object to be shot or recorded, this instruction is inputted to a system control part 24 and the system control part 24 informs a solid-state image pick-up element 10 of the instruction for shooting or recording the object.

When the solid-state image pick-up element 10 receives the instruction for shooting or recording the object, a control part 40 considers it as a start trigger to supply reset pulses to reset gates RG of reset transistors RET of all pixel parts 100. Thus, unnecessary electric charges respectively stored in photoelectric conversion parts 11 of the pixel parts 100 are discharged to the drains of the reset transistors RET.

After the reset operation is completed, the system control part 24 outputs an instruction to a light source driving part 21 to emit an R light, a G light and a B light from an LED 1a, an LED 1b and an LED 1c at the same time. In FIG. 8, the R light, the G light and the B light are emitted at the same time after a little time when the reset pulses are supplied, however, the R, G and B lights may be emitted at the same time as the completion of the reset operation. Further, the R light, the G light and the B light may not be emitted at the same time and may be continuously emitted by slightly shifting timings.

The R light, the G light and the B light are emitted, for instance, only during an exposure period set by the endoscope device. During the emission of the lights, in the pixel parts 100 of the solid-state image pick-up element 10 respectively, lights incident from the object are incident on the photoelectric conversion parts 11 and the electric charges corresponding to the R light, the G light and the B light are generated and stored.

After the exposure period is finished, the control part 40 supplies writing pulses to the writing control gates WCG1 of all the pixel parts 100 to store the electric charges generated in the photoelectric conversion parts 11 during the exposure period in floating gates FG1. In supplying the writing pulses, either a method for starting the supply of the writing pulses at the same time as the completion of the exposure period or a method for starting the supply of the writing pulses at the same time as the start of the exposure period and completing the supply of the writing pulses at the same time as the completion of the exposure period may be employed.

In accordance with the supply of the writing pulses, as shown in FIG. 9, the electric charges (the electric charges by the R light, the G light and the B light, shown by “RGB (Y)” in the drawing) generated in the pixel parts 100 are stored respectively in the floating gates FG1 of the pixel parts 100. The electric charges stored in the floating gates FG1 are electric charges including all components of the R light, the G light and the B light. A luminance signal forming color image data is formed by adding a signal component corresponding to the R light, a signal component corresponding to the G light and a signal component corresponding to the B light which are weighted by a prescribed coefficient. An amount of emission of the R light, the G light and the B light is set to the prescribed coefficient, so that signals corresponding to the electric charges stored in the floating gates FG1 may be treated as the luminance signals. Thus, in the endoscope device of the first modified example, the amount of emission of the R light, the G light and the B light is set to a value corresponding to the coefficient employed when the luminance signal is formed.

When the storage of the electric charges in the floating gates FG1 is finished, the control part 40 supplies again reset pulses to the reset gates RG of the reset transistors RET of all the pixel parts 100. Thus, remaining electric charges which are hardly injected to the floating gates FG1 from the photoelectric conversion parts 11 to be left are discharged to the drains of the reset transistors RET.

After a second reset operation is completed, the system control part 24 outputs an instruction to the light source driving part 21 to emit the R light from the LED 1a. In FIG. 8, the R light is emitted after a little time when the reset pulses are supplied, however, the R light may be emitted at the same time as the completion of the reset operation.

The R light is emitted, for instance, only during an exposure period set by the endoscope device. During the emission of the R light, in the pixel parts 100 of the solid-state image pick-up element 10 respectively, lights incident from the object are incident on the photoelectric conversion parts 11 and the electric charges corresponding to the R light are generated and stored.

After the exposure period is finished, the control part 40 supplies writing pulses to the writing control gates WCG2 of all the pixel parts 100 to store the electric charges generated in the photoelectric conversion parts 11 during the exposure period in floating gates FG2. In supplying the writing pulses, either a method for starting the supply of the writing pulses at the same time as the completion of the exposure period or a method for starting the supply of the writing pulses at the same time as the start of the exposure period and completing the supply of the writing pulses at the same time as the completion of the exposure period may be employed.

In accordance with the supply of the writing pulses, as shown in FIG. 9, the electric charges (the electric charges by the R light, shown by “R” in the drawing) generated in the pixel parts 100 are stored respectively in the floating gates FG2 of the pixel parts 100.

When the storage of the electric charges in the floating gates FG2 is finished, the control part 40 supplies again reset pulses to the reset gates RG of the reset transistors RET of all the pixel parts 100. Thus, remaining electric charges which are hardly injected to the floating gates FG2 from the photoelectric conversion parts 11 to be left are discharged to the drains of the reset transistors RET.

After a third reset operation is completed, the system control part 24 outputs an instruction to the light source driving part 21 to emit the B light from the LED 1c. In FIG. 8, the B light is emitted after a little time when the reset pulses are supplied, however, the B light may be emitted at the same time as the completion of the reset operation.

The B light is emitted, for instance, only during an exposure period set by the endoscope device. During the emission of the B light, in the pixel parts 100 of the solid-state image pick-up element 10 respectively, lights incident from the object are incident on the photoelectric conversion parts 11 and the electric charges corresponding to the B light are generated and stored.

After the exposure period is finished, the control part 40 supplies writing pulses to the writing control gates WCG3 of all the pixel parts 100 to store the electric charges generated in the photoelectric conversion parts 11 during the exposure period in floating gates FG3. In supplying the writing pulses, either a method for starting the supply of the writing pulses at the same time as the completion of the exposure period or a method for starting the supply of the writing pulses at the same time as the start of the exposure period and completing the supply of the writing pulses at the same time as the completion of the exposure period may be employed.

In accordance with the supply of the writing pulses, as shown in FIG. 9, the electric charges (the electric charges by the B light, shown by “B” in the drawing) generated in the pixel parts 100 are stored respectively in the floating gates FG3 of the pixel parts 100.

After the electric charges are completely stored in the floating gates FG3, the control part 40 pre-charges the drains of the reading transistors RT1 of the pixel parts 100 respectively in a first line to begin to apply a lamp wave form voltage to the reading control gates RCG1 of the pixel parts 100 of the first line. Then, count values corresponding to the value of the lamp wave form voltage when the drain potentials of the reading transistors RT1 of the first line drop are respectively held in reading circuits 20 and the count values are outputted from an output amplifier 60 as image pick-up signals. The control part 40 carries out a similar driving operation in lines after a second line to output first image pick-up signals (luminance signals Y) corresponding to the electric charges stored in the floating gates FG1 of all lines.

Then, the control part 40 pre-charges the drains of the reading transistors RT2 of the pixel parts 100 respectively in the first line to begin to apply the lamp wave form voltage to the reading control gates RCG2 of the pixel parts 100 of the first line. Then, count values corresponding to the value of the lamp wave form voltage when the drain potentials of the reading transistors RT2 of the first line drop are respectively held in reading circuits 20 and the count values are outputted from the output amplifier 60 as image pick-up signals. The control part 40 carries out a similar driving operation in lines after a second line to output second image pick-up signals (R signals) corresponding to the electric charges stored in the floating gates FG2 of all lines.

Then, the control part 40 pre-charges the drains of the reading transistors RT3 of the pixel parts 100 respectively in the first line to begin to apply the lamp wave form voltage to the reading control gates RCG3 of the pixel parts 100 of the first line. Then, count values corresponding to the value of the lamp wave form voltage when the drain potentials of the reading transistors RT3 of the first line drop are respectively held in reading circuits 20 and the count values are outputted from the output amplifier 60 as image pick-up signals. The control part 40 carries out a similar driving operation in lines after a second line to output third image pick-up signals (B signals) corresponding to the electric charges stored in the floating gates FG3 of all lines.

After the third image pick-up signals are outputted, the control part 40 sets the potentials of the writing control gates WCG1, WCG2 and WCG3 and the reading control gates RCG1, RCG2 and RCG3 of all the pixel parts 100 to—Vcc and the potential of a semiconductor substrate to Vcc. Thus, the electric charges stored in the floating gates FG1, FG2 and FG3 are drawn out to the semiconductor substrate and erased.

The above-described operations are carried out within one frame period.

A signal processing part 123 forms color image data by the luminance signals Y, the R signals and the B signals respectively outputted from the pixel parts 100 of the solid-state image pick-up element 10. Specifically, a color difference signal Cr is formed from the luminance signal Y and the R signal and a color difference signal Cb is formed from the luminance signal Y and the B signal to form the color image data of a JPEG form composed of a YC signal. By a color image based on the color image data, the same state as that the object is observed by the naked eye may be reproduced on a display part 22.

As described above, according to the endoscope device of the first modified example, a calculation for forming the luminance signal may not be required. Therefore, a calculating time until the image data is formed may be shortened, and a frame rate at the time of shooting or recording a moving image may be improved. Further, since the luminance signal is determined by color characteristics of a light source 1 (spectral characteristics of the colors of R, G and B respectively), an image high in its fidelity may be obtained and a diagnosis of high accuracy may be realized.

### Second Modified Example

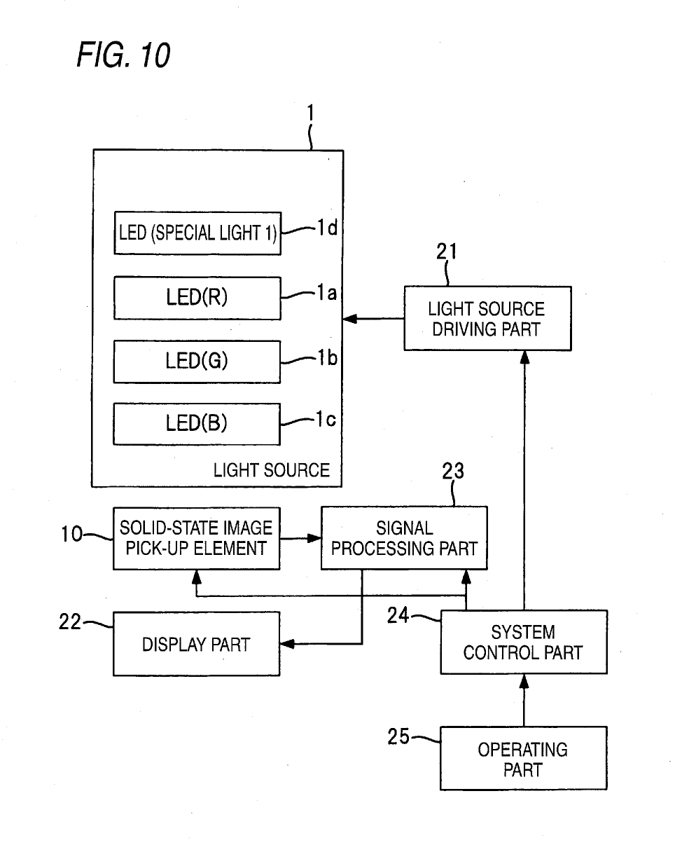

FIG. 10 is a diagram showing a schematic structure of an endoscope device of a second modified example. The endoscope device shown in FIG. 10 has a structure in which an LED 1d for emitting a special light 1 is added to the light source 1 of the endoscope device shown in FIG. 1.

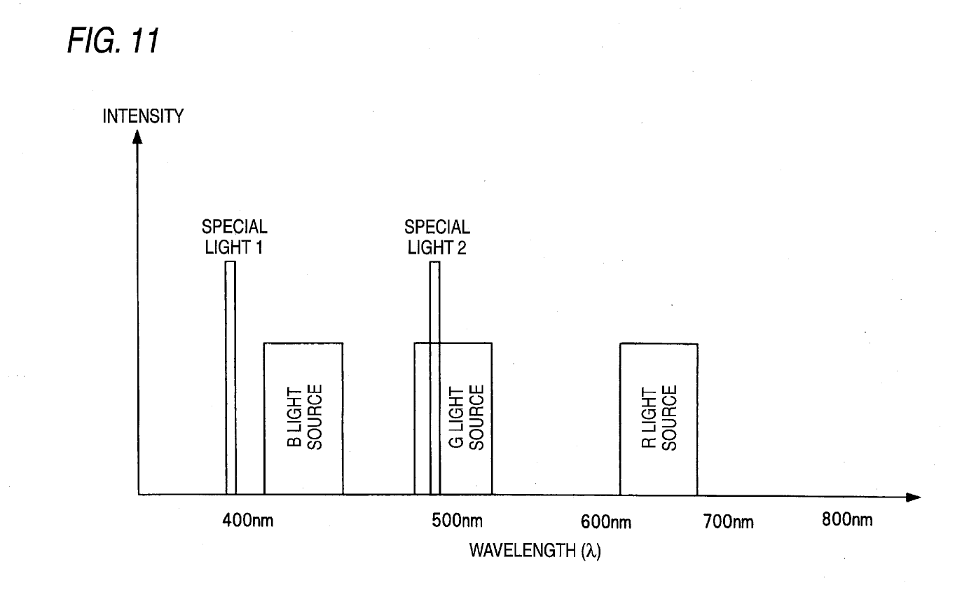

The special light 1 is a light necessary for a person to identify biological information that cannot be identified by RGB lights (white color light). For instance, as shown in FIG. 11, the special light 1 has a light including a bright line in a specific wavelength located outside the wavelength areas of the R light, the G light and the B light. The specific wavelength of the special light 1 may be arbitrarily determined depending on biological information desired to be observed. Various kinds of wavelengths may be set, for instance, a wavelength for lighting an object to definitely recognize whether or not a red color (hemoglobin) appears, a wavelength for lighting an object to definitely recognize whether or not there is an independent fluorescence, a wavelength for lighting an object to definitely recognize a blood vessel in the depth of the object or the like.

When the light of the specific wavelength is applied to a certain object, the object emits an excitation light of a wavelength different from the specific wavelength and an image by the excitation light may be occasionally desired to be observed. In order to detect the excitation light, a light having a wavelength of emitted light that generates the excitation light from the object may be set as the special light.

For instance, when a light having a wavelength of 400 nm is desired to be applied to the object to detect the light reflected from the object, as the special light 1, a light having the wavelength of an emitted light in the wavelength of 400 nm may be set to be emitted. Further, for instance, when a light having a wavelength of 650 nm is applied to the object, an excitation light having a wavelength of 680 nm is supposed to be emitted. When the excitation light is desired to be detected, as the special light 1, a light having the wavelength of an emitted light in the wavelength of 650 nm may be set to be emitted. Now, an operation of the endoscope device of the second modified embodiment will be described below.

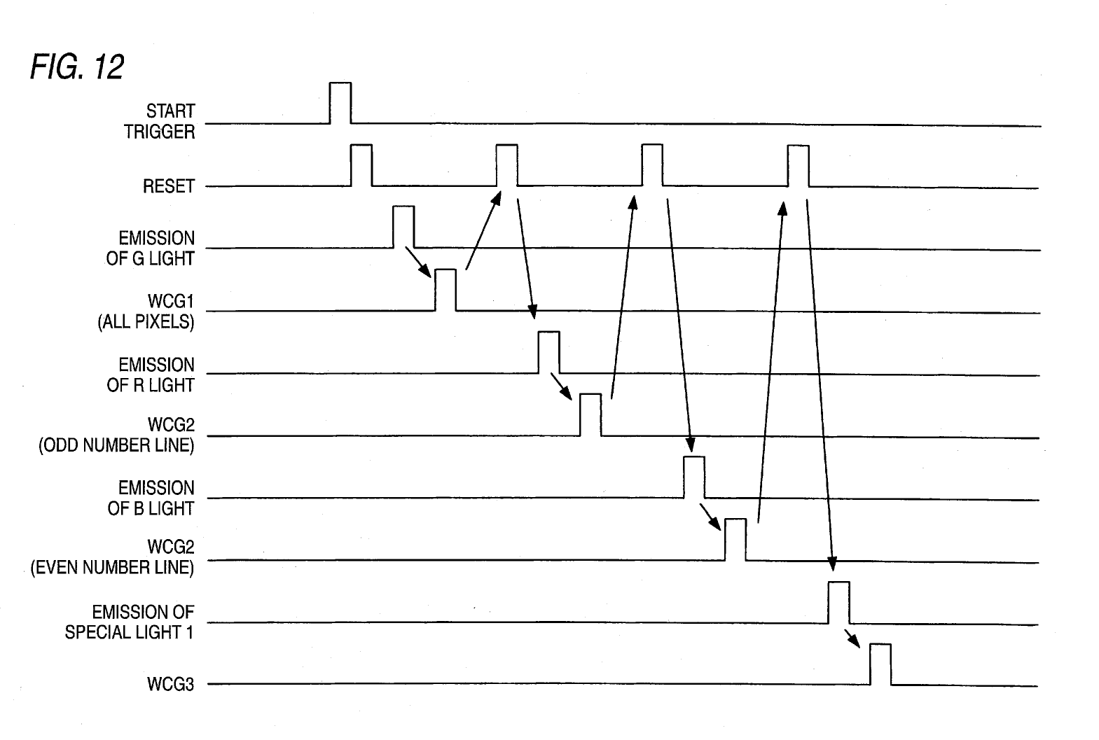

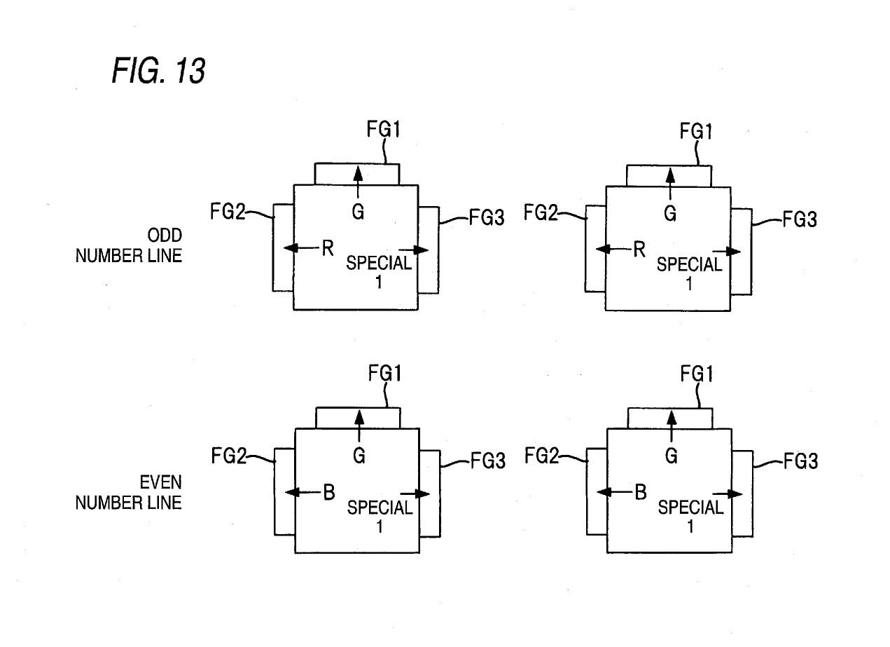

FIG. 12 is a timing chart for explaining the operation of the endoscope device of the second modified example. FIG. 13 is a schematic view for explaining the operation of the endoscope device of the second modified example. In FIG. 13, four pixel parts of 2 rows×2 columns in total are schematically shown.

When an operating part 25 is operated to instruct an object to be shot or recorded, this instruction is inputted to a system control part 24 and the system control part 24 informs a solid-state image pick-up element 10 of the instruction for shooting or recording the object.

When the solid-state image pick-up element 10 receives the instruction for shooting or recording the object, a control part 40 considers it as a start trigger to supply reset pulses to reset gates RG of reset transistors RET of all pixel parts 100. Thus, unnecessary electric charges respectively stored in photoelectric conversion parts 11 of the pixel parts 100 are discharged to the drains of the reset transistors RET.

After a reset operation is completed, the system control part 24 outputs an instruction to a light source driving part 21 to emit a G light from an LED 1b. In FIG. 12, the G light is emitted after a little time when the reset pulses are supplied, however, the G light may be emitted at the same time as the completion of the reset operation.

The G light is emitted, for instance, only during an exposure period set by the endoscope device. During the emission of the G light, in the pixel parts 100 of the solid-state image pick-up element 10 respectively, lights incident from the object are incident on the photoelectric conversion parts 11 and the electric charges corresponding to the G light are generated and stored therein.

After the exposure period is finished, the control part 40 supplies writing pulses to the writing control gates WCG1 of all the pixel parts 100 to store the electric charges generated in the photoelectric conversion parts 11 during the exposure period in floating gates FG1. In supplying the writing pulses, either a method for starting the supply of the writing pulses at the same time as the completion of the exposure period or a method for starting the supply of the writing pulses at the same time as the start of the exposure period and completing the supply of the writing pulses at the same time as the completion of the exposure period may be employed.

In accordance with the supply of the writing pulses, as shown in FIG. 13, the electric charges (the electric charges by the G light, shown by “G” in the drawing) generated in the pixel parts 100 are stored respectively in the floating gates FG1 of the pixel parts 100.

When the storage of the electric charges in the floating gates FG1 is finished, the control part 40 supplies again reset pulses to the reset gates RG of the reset transistors RET of all the pixel parts 100. Thus, remaining electric charges which are hardly injected to the floating gates FG1 from the photoelectric conversion parts 11 to be left are discharged to the drains of the reset transistors RET.

After a second reset operation is completed, the system control part 24 outputs an instruction to the light source driving part 21 to emit an R light from an LED 1a. In FIG. 12, the R light is emitted after a little time when the reset pulses are supplied, however, the R light may be emitted at the same time as the completion of the reset operation.

The R light is emitted, for instance, only during an exposure period set by the endoscope device. During the emission of the R light, in the pixel parts 100 of the solid-state image pick-up element 10 respectively, lights incident from the object are incident on the photoelectric conversion parts 11 and the electric charges corresponding to the R light are generated and stored therein.

After the exposure period is finished, the control part 40 supplies writing pulses to the writing control gates WCG2 of the pixel parts 100 of odd number lines to store the electric charges generated in the photoelectric conversion parts 11 during the exposure period in floating gates FG2. In supplying the writing pulses, either a method for starting the supply of the writing pulses at the same time as the completion of the exposure period or a method for starting the supply of the writing pulses at the same time as the start of the exposure period and completing the supply of the writing pulses at the same time as the completion of the exposure period may be employed.

In accordance with the supply of the writing pulses, as shown in FIG. 13, the electric charges (the electric charges by the R light, shown by “R” in the drawing) generated in the pixel parts 100 are stored respectively only in the floating gates FG2 of the pixel parts 100 of the odd number lines.

When the storage of the electric charges in the floating gates FG2 of the pixel parts 100 of the odd number lines is finished, the control part 40 supplies again reset pulses to the reset gates RG of the reset transistors RET of all the pixel parts 100. Thus, remaining electric charges which are hardly injected to the floating gates FG2 from the photoelectric conversion parts 11 to be left are discharged to the drains of the reset transistors RET.

After a third reset operation is completed, the system control part 24 outputs an instruction to the light source driving part 21 to emit a B light from an LED 1c. In FIG. 12, the B light is emitted after a little time when the reset pulse is supplied, however, the B light may be emitted at the same time as the completion of the reset operation.

The B light is emitted, for instance, only during an exposure period set by the endoscope device. During the emission of the B light, in the pixel parts 100 of the solid-state image pick-up element 10 respectively, lights incident from the object are incident on the photoelectric conversion parts 11 and the electric charges corresponding to the B light are generated and stored therein.

After the exposure period is finished, the control part 40 supplies writing pulses to the writing control gates WCG2 of the pixel parts 100 of even number lines to store the electric charges generated in the photoelectric conversion parts 11 during the exposure period in floating gates FG2. In supplying the writing pulses, either a method for starting the supply of the writing pulses at the same time as the completion of the exposure period or a method for starting the supply of the writing pulses at the same time as the start of the exposure period and completing the supply of the writing pulses at the same time as the completion of the exposure period may be employed.

In accordance with the supply of the writing pulses, as shown in FIG. 13, the electric charges (the electric charges by the B light, shown by “B” in the drawing) generated in the pixel parts 100 are stored respectively only in the floating gates FG2 of the pixel parts 100 of the even number lines.

When the storage of the electric charges in the floating gates FG2 of the pixel parts 100 of the even number lines is finished, the control part 40 supplies again reset pulses to the reset gates RG of the reset transistors RET of all the pixel parts 100. Thus, remaining electric charges which are hardly injected to the floating gates FG2 from the photoelectric conversion parts 11 to be left are discharged to the drains of the reset transistors RET.

After a fourth reset operation is completed, the system control part 24 outputs an instruction to the light source driving part 21 to emit the special light 1 from the LED 1d. In FIG. 12, the special light 1 is emitted after a little time when the reset pulses are supplied, however, the special light 1 may be emitted at the same time as the completion of the reset operation.

The special light 1 is emitted, for instance, only during an exposure period set by the endoscope device. During the emission of the special light 1, in the pixel parts 100 of the solid-state image pick-up element 10 respectively, lights incident from the object are incident on the photoelectric conversion parts 11 and the electric charges corresponding to the special light 1 are generated and stored therein.

After the exposure period is finished, the control part 40 supplies writing pulses to the writing control gates WCG3 of all the pixel parts 100 to store the electric charges generated in the photoelectric conversion parts 11 during the exposure period in floating gates FG3. In supplying the writing pulses, either a method for starting the supply of the writing pulses at the same time as the completion of the exposure period or a method for starting the supply of the writing pulses at the same time as the start of the exposure period and completing the supply of the writing pulses at the same time as the completion of the exposure period may be employed.

In accordance with the supply of the writing pulses, as shown in FIG. 13, the electric charges (the electric charges by the special light 1, shown by “special 1” in the drawing) generated in the pixel parts 100 are stored respectively in the floating gates FG3 of the pixel parts 100.

After the electric charges are completely stored in the floating gates FG3, the control part 40 pre-charges the drains of the reading transistors RT1 of the pixel parts 100 respectively in a first line to begin to apply a lamp wave form voltage to the reading control gates RCG1 of the pixel parts 100 of the first line. Then, count values corresponding to the value of the lamp wave form voltage when the drain potentials of the reading transistors RT1 of the first line drop are respectively held in reading circuits 20 and the count values are outputted from an output amplifier 60 as image pick-up signals. The control part 40 carries out a similar driving operation in lines after a second line to output first image pick-up signals (G signals) corresponding to the electric charges stored in the floating gates FG1 of all lines.

Then, the control part 40 pre-charges the drains of the reading transistors RT2 of the pixel parts 100 respectively in the first line to begin to apply the lamp wave form voltage to the reading control gates RCG2 of the pixel parts 100 of the first line. Then, count values corresponding to the value of the lamp wave form voltage when the drain potentials of the reading transistors RT2 of the first line drop are respectively held in the reading circuits 20 and the count values are outputted from the output amplifier 60 as image pick-up signals. The control part 40 carries out a similar driving operation in lines after a second line to output second image pick-up signals (R signals and B signals) corresponding to the electric charges stored in the floating gates FG2 of all lines.

Then, the control part 40 pre-charges the drains of the reading transistors RT3 of the pixel parts 100 respectively in the first line to begin to apply the lamp wave form voltage to the reading control gates RCG3 of the pixel parts 100 of the first line. Then, count values corresponding to the value of the lamp wave form voltage when the drain potentials of the reading transistors RT3 of the first line drop are respectively held in the reading circuits 20 and the count values are outputted from the output amplifier 60 as image pick-up signals. The control part 40 carries out a similar driving operation in lines after a second line to output third image pick-up signals (special light 1 signals) corresponding to the electric charges stored in the floating gates FG3 of all lines.

After the third image pick-up signals are outputted, the control part 40 sets the potentials of the writing control gates WCG1, WCG2 and WCG3 and the reading control gates RCG1, RCG2 and RCG3 of all the pixel parts 100 to—Vcc and the potential of a semiconductor substrate to Vcc. Thus, the electric charges stored in the floating gates FG1, FG2 and FG3 are drawn out to the semiconductor substrate and erased.

The above-described operations are carried out within one frame period.

A signal processing part 23 forms color image data and monochromatic image data by the G signals, the R signals or the B signals and the special light 1 signals outputted respectively from the pixel parts 100 of the image pick-up element 10. Specifically, the R signals and the B signals that are not obtained from the pixel parts 100 are interpolated by using R signals and B signals obtained from the pixel parts 100 in the periphery of the pixel parts 100 to form the R signal, the G signal and the B signal for one pixel part 100. A luminance signal and a color difference signal are formed from these signals to form the color image data. Further, the monochromatic image data is formed from the special light 1 signal.

As described above, according to the endoscope device of the second modified example, the monochromatic image data by the special light 1 may be obtained as well as the color image data by carrying out a shooting or recording operation once. The color image data is formed by using the floating gates FG1 and FG2 and the monochromatic image data is formed by using the floating gates FG3. A little time is necessary between the storage of the electric charges for forming the color image data and the storage of the electric charges for forming the monochromatic image data. Therefore, even when the object to be shot or recorded moves, a possibility is low that the object to be shot or recorded may shift between the color image data and the monochromatic image data. As a result, for the same object to be shot or recorded, an image may be observed under different conditions, so that a proper diagnosis may be realized.

In the above explanation, the electric charges corresponding to the R light are stored in the floating gates FG2 of the odd number lines, and the electric charges corresponding to the B light are stored in the floating gates FG2 of the even number lines. However, the above-described relation may be reversed. Further, the electric charges corresponding to the R light may be stored in the floating gates FG2 half as many as all the floating gates FG2, and the electric charges corresponding to the B light may be stored in remaining floating gates FG2 half as many as all the floating gates FG2. Thus, the floating gates FG2 may not be divided into the floating gates FG2 of the odd number lines and the floating gates FG2 of the even number lines.

### Third Modified Example

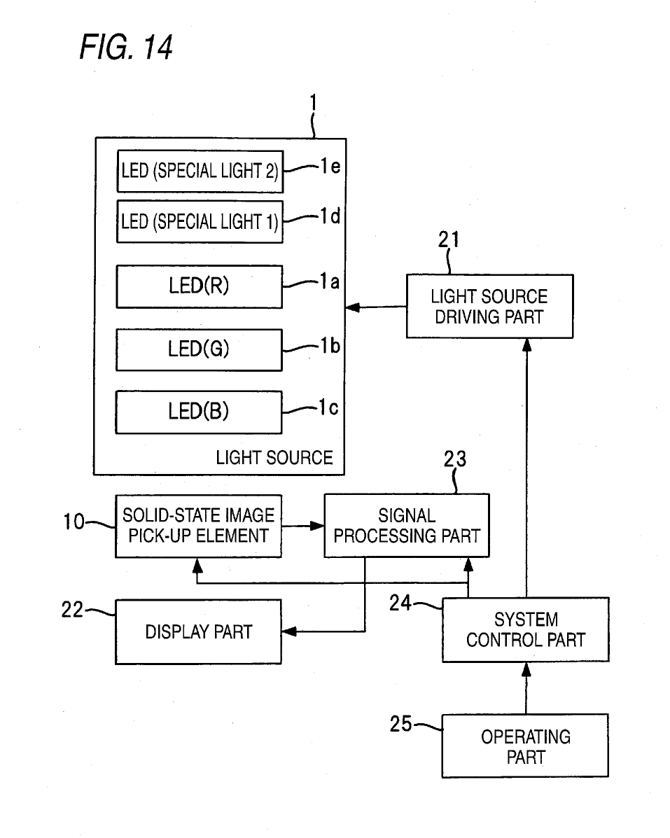

FIG. 14 is a diagram showing a schematic structure of an endoscope device of a third modified example. The endoscope device shown in FIG. 14 has a structure in which an LED 1e for emitting a special light 2 is added to the light source 1 of the endoscope device shown in FIG. 10.

The special light 2 is a light necessary for a person to identify a part that cannot be identified by RGB lights (white color light) like the special light 1. For instance, as shown in FIG. 11, the special light 2 is a light including a bright line in a specific wavelength located within the wavelength areas of a G light. The specific wavelength of the special light 2 may be arbitrarily determined depending on biological information desired to be observed like the special light 1. However, the special light 2 has the bright line in the wavelength different from that of the special light 1.

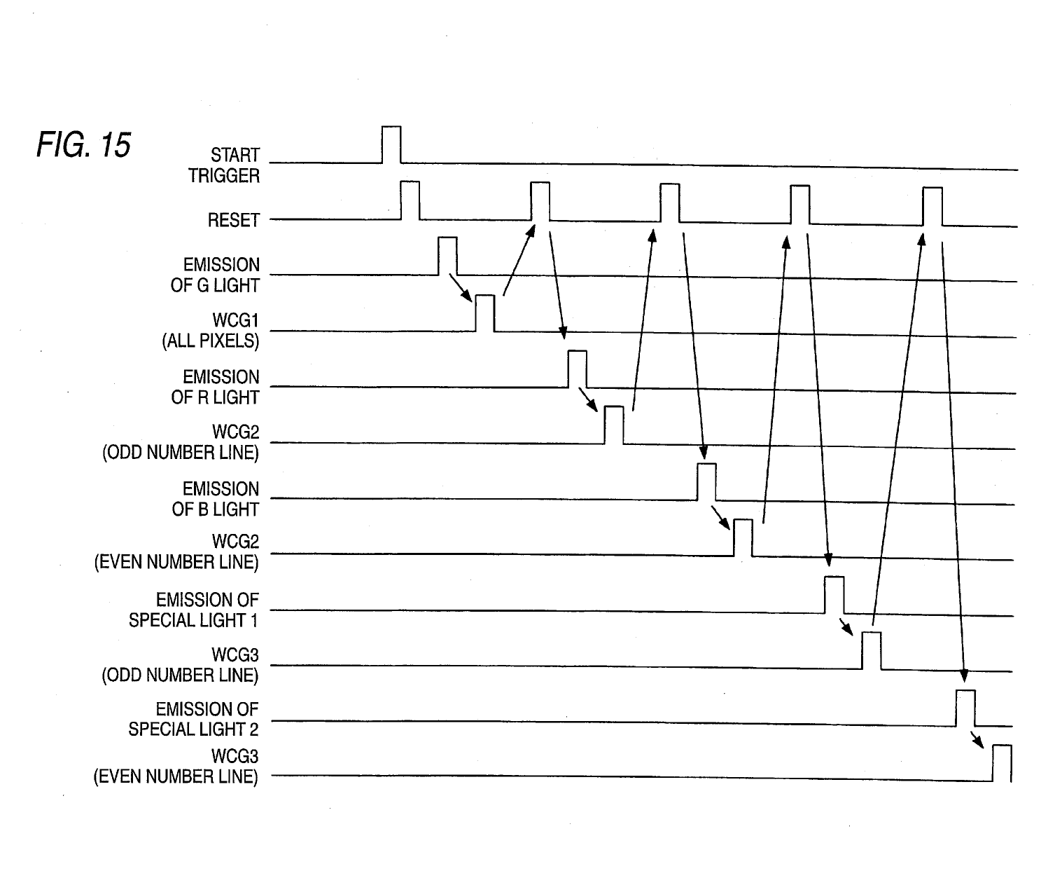

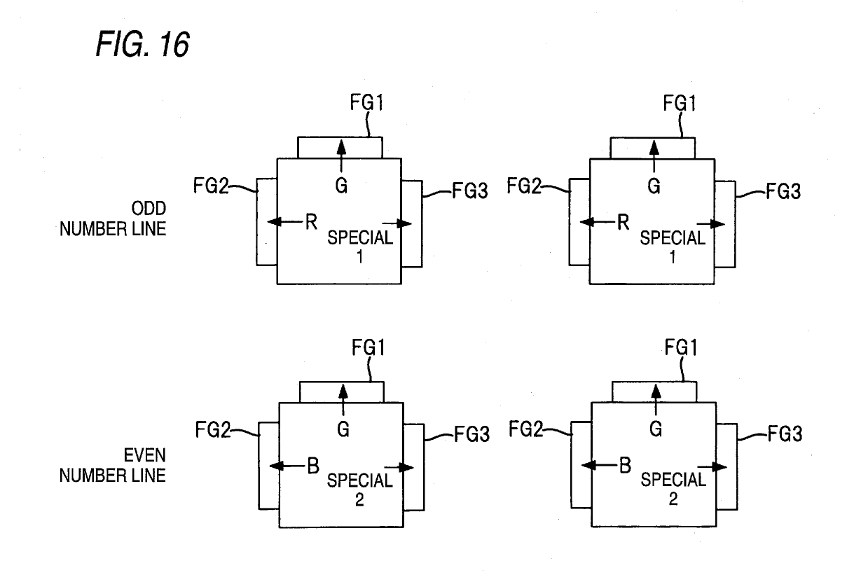

FIG. 15 is a timing chart for explaining the operation of the endoscope device of the third modified example. FIG. 16 is a schematic view for explaining the operation of the endoscope device of the third modified example. In FIG. 16, four pixel parts of 2 rows×2 columns in total are schematically shown.

When an operating part 25 is operated to instruct an object to be shot or recorded, this instruction is inputted to a system control part 24 and the system control part 24 informs a solid-state image pick-up element 10 of the instruction for shooting or recording the object.

When the solid-state image pick-up element 10 receives the instruction for shooting or recording the object, a control part 40 considers it as a start trigger to supply reset pulses to reset gates RG of reset transistors RET of all pixel parts 100. Thus, unnecessary electric charges respectively stored in photoelectric conversion parts 11 of the pixel parts 100 are discharged to the drains of the reset transistors RET.

After a reset operation is completed, the system control part 24 outputs an instruction to a light source driving part 21 to emit a G light from an LED 1b. In FIG. 15, the G light is emitted after a little time when the reset pulses are supplied, however, the G light may be emitted at the same time as the completion of the reset operation.

The G light is emitted, for instance, only during an exposure period set by the endoscope device. During the emission of the G light, in the pixel parts 100 of the solid-state image pick-up element 10 respectively, lights incident from the object are incident on the photoelectric conversion parts 11 and the electric charges corresponding to the G light are generated and stored therein.

After the exposure period is finished, the control part 40 supplies writing pulses to the writing control gates WCG1 of all the pixel parts 100 to store the electric charges generated in the photoelectric conversion parts 11 during the exposure period in floating gates FG1. In supplying the writing pulses, either a method for starting the supply of the writing pulses at the same time as the completion of the exposure period or a method for starting the supply of the writing pulses at the same time as the start of the exposure period and completing the supply of the writing pulses at the same time as the completion of the exposure period may be employed.

In accordance with the supply of the writing pulses, as shown in FIG. 16, the electric charges (the electric charges by the G light, shown by “G” in the drawing) generated in the pixel parts 100 are stored respectively in the floating gates FG1 of the pixel parts 100.

When the storage of the electric charges in the floating gates FG1 is finished, the control part 40 supplies again reset pulses to the reset gates RG of the reset transistors RET of all the pixel parts 100. Thus, remaining electric charges which are hardly injected to the floating gates FG1 from the photoelectric conversion parts 11 to be left are discharged to the drains of the reset transistors RET.

After a second reset operation is completed, the system control part 24 outputs an instruction to the light source driving part 21 to emit an R light from an LED 1a. In FIG. 15, the R light is emitted after a little time when the reset pulses are supplied, however, the R light may be emitted at the same time as the completion of the reset operation.

The R light is emitted, for instance, only during an exposure period set by the endoscope device. During the emission of the R light, in the pixel parts 100 of the solid-state image pick-up element 10 respectively, lights incident from the object are incident on the photoelectric conversion parts 11 and the electric charges corresponding to the R light are generated and stored therein.

After the exposure period is finished, the control part 40 supplies writing pulses to the writing control gates WCG2 of the pixel parts 100 of odd number lines to store the electric charges generated in the photoelectric conversion parts 11 during the exposure period in floating gates FG2. In supplying the writing pulses, either a method for starting the supply of the writing pulses at the same time as the completion of the exposure period or a method for starting the supply of the writing pulses at the same time as the start of the exposure period and completing the supply of the writing pulses at the same time as the completion of the exposure period may be employed.

In accordance with the supply of the writing pulses, as shown in FIG. 16, the electric charges (the electric charges by the R light, shown by “R” in the drawing) generated in the pixel parts 100 are stored respectively only in the floating gates FG2 of the pixel parts 100 of the odd number lines.

When the storage of the electric charges in the floating gates FG2 of the pixel parts 100 of the odd number lines is finished, the control part 40 supplies again reset pulses to the reset gates RG of the reset transistors RET of all the pixel parts 100. Thus, remaining electric charges which are hardly injected to the floating gates FG2 from the photoelectric conversion parts 11 to be left are discharged to the drains of the reset transistors RET.

After a third reset operation is completed, the system control part 24 outputs an instruction to the light source driving part 21 to emit a B light from an LED 1c. In FIG. 15, the B light is emitted after a little time when the reset pulses are supplied, however, the B light may be emitted at the same time as the completion of the reset operation.

The B light is emitted, for instance, only during an exposure period set by the endoscope device. During the emission of the B light, in the pixel parts 100 of the solid-state image pick-up element 10 respectively, lights incident from the object are incident on the photoelectric conversion parts 11 and the electric charges corresponding to the B light are generated and stored therein.

After the exposure period is finished, the control part 40 supplies writing pulses to the writing control gates WCG2 of the pixel parts 100 of even number lines to store the electric charges generated in the photoelectric conversion parts 11 during the exposure period in floating gates FG2. In supplying the writing pulses, either a method for starting the supply of the writing pulses at the same time as the completion of the exposure period or a method for starting the supply of the writing pulses at the same time as the start of the exposure period and completing the supply of the writing pulses at the same time as the completion of the exposure period may be employed.

In accordance with the supply of the writing pulses, as shown in FIG. 16, the electric charges (the electric charges by the B light, shown by “B” in the drawing) generated in the pixel parts 100 are stored respectively only in the floating gates FG2 of the pixel parts 100 of the even number lines.

When the storage of the electric charges in the floating gates FG2 of the pixel parts 100 of the even number lines is finished, the control part 40 supplies again reset pulses to the reset gates RG of the reset transistors RET of all the pixel parts 100. Thus, remaining electric charges which are hardly injected to the floating gates FG2 from the photoelectric conversion parts 11 to be left are discharged to the drains of the reset transistors RET.

After a fourth reset operation is completed, the system control part 24 outputs an instruction to the light source driving part 21 to emit the special light 1 from an LED 1d. In FIG. 15, the special light 1 is emitted after a little time when the reset pulses are supplied, however, the special light 1 may be emitted at the same time as the completion of the reset operation.

The special light 1 is emitted, for instance, only during an exposure period set by the endoscope device. During the emission of the special light 1, in the pixel parts 100 of the solid-state image pick-up element 10 respectively, lights incident from the object are incident on the photoelectric conversion parts 11 and the electric charges corresponding to the special light 1 are generated and stored therein.

After the exposure period is finished, the control part 40 supplies writing pulses to the writing control gates WCG3 of the pixel parts 100 of the odd number lines to store the electric charges generated in the photoelectric conversion parts 11 during the exposure period in floating gates FG3. In supplying the writing pulses, either a method for starting the supply of the writing pulses at the same time as the completion of the exposure period or a method for starting the supply of the writing pulses at the same time as the start of the exposure period and completing the supply of the writing pulses at the same time as the completion of the exposure period may be employed.

In accordance with the supply of the writing pulses, as shown in FIG. 16, the electric charges (the electric charges by the special light 1, shown by “special 1” in the drawing) generated in the pixel parts 100 are stored respectively only in the floating gates FG3 of the pixel parts 100 of the odd number lines.

When the storage of the electric charges in the floating gates FG3 of the pixel parts 100 of the odd number lines is finished, the control part 40 supplies again reset pulses to the reset gates RG of the reset transistors RET of all the pixel parts 100. Thus, remaining electric charges which are hardly injected to the floating gates FG3 from the photoelectric conversion parts 11 to be left are discharged to the drains of the reset transistors RET.

After a fifth reset operation is completed, the system control part 24 outputs an instruction to the light source driving part 21 to emit the special light 2 from the LED 1e. In FIG. 15, the special light 2 is emitted after a little time when the reset pulses are supplied, however, the special light 2 may be emitted at the same time as the completion of the reset operation.

The special light 2 is emitted, for instance, only during an exposure period set by the endoscope device. During the emission of the special light 2, in the pixel parts 100 of the solid-state image pick-up element 10 respectively, lights incident from the object are incident on the photoelectric conversion parts 11 and the electric charges corresponding to the special light 2 are generated and stored therein.

After the exposure period is finished, the control part 40 supplies writing pulses to the writing control gates WCG3 of the pixel parts 100 of the even number lines to store the electric charges generated in the photoelectric conversion parts 11 during the exposure period in floating gates FG3. In supplying the writing pulses, either a method for starting the supply of the writing pulses at the same time as the completion of the exposure period or a method for starting the supply of the writing pulses at the same time as the start of the exposure period and completing the supply of the writing pulses at the same time as the completion of the exposure period may be employed.

In accordance with the supply of the writing pulses, as shown in FIG. 16, the electric charges (the electric charges by the special light 2, shown by “special 2” in the drawing) generated in the pixel parts 100 are stored respectively only in the floating gates FG3 of the pixel parts 100 of the even number lines.

After the electric charges are completely stored in the floating gates FG3, the control part 40 pre-charges the drains of the reading transistors RT1 of the pixel parts 100 respectively in a first line to begin to apply a lamp wave form voltage to the reading control gates RCG1 of the pixel parts 100 of the first line. Then, count values corresponding to the value of the lamp wave form voltage when the drain potentials of the reading transistors RT1 of the first line drop are respectively held in reading circuits 20 and the count values are outputted from an output amplifier 60 as image pick-up signals. The control part 40 carries out a similar driving operation in lines after a second line to output first image pick-up signals (G signals) corresponding to the electric charges stored in the floating gates FG1 of all lines.

Then, the control part 40 pre-charges the drains of the reading transistors RT2 of the pixel parts 100 respectively in the first line to begin to apply the lamp wave form voltage to the reading control gates RCG2 of the pixel parts 100 of the first line. Then, count values corresponding to the value of the lamp wave form voltage when the drain potentials of the reading transistors RT2 of the first line drop are respectively held in the reading circuits 20 and the count values are outputted from the output amplifier 60 as image pick-up signals. The control part 40 carries out a similar driving operation in lines after a second line to output second image pick-up signals (R signals and B signals) corresponding to the electric charges stored in the floating gates FG2 of all lines.

Then, the control part 40 pre-charges the drains of the reading transistors RT3 of the pixel parts 100 respectively in the first line to begin to apply the lamp wave form voltage to the reading control gates RCG3 of the pixel parts 100 of the first line. Then, count values corresponding to the value of the lamp wave form voltage when the drain potentials of the reading transistors RT3 of the first line drop are respectively held in the reading circuits 20 and the count values are outputted from the output amplifier 60 as image pick-up signals. The control part 40 carries out a similar driving operation in lines after a second line to output third image pick-up signals (special light 1 signals and special light 2 signals) corresponding to the electric charges stored in the floating gates FG3 of all lines.

After the third image pick-up signals are outputted, the control part 40 sets the potentials of the writing control gates WCG1, WCG2 and WCG3 and the reading control gates RCG1, RCG2 and RCG3 of all the pixel parts 100 to—Vcc and the potential of a semiconductor substrate to Vcc. Thus, the electric charges stored in the floating gates FG1, FG2 and FG3 are drawn out to the semiconductor substrate and erased.

The above-described operations are carried out within one frame period.

A signal processing part 23 forms color image data and two monochromatic image data by the G signals, the R signals or the B signals and the special light 1 signals or the special light 2 signals outputted respectively from the pixel parts 100 of the image pick-up element 10. Specifically, the R signals and the B signals that are not obtained from the pixel parts 100 are interpolated by using R signals and B signals obtained from the pixel parts 100 in the periphery of the pixel parts 100 to form the R signal, the G signal and the B signal for one pixel part 100 and form the color image data. Further, the special light 1 signals or the special light 2 signals that are not obtained from the pixel parts 100 are interpolated by using special light 1 signals and special light 2 signals obtained from the pixel parts 100 in the periphery of the pixel parts 100 to form the special light 1 signal and the special light 2 signal for one pixel part 100 and form the two monochromatic image data. The special light 1 signals and the special light 2 signals may not be interpolated to directly form the monochromatic image data of the pixel parts half as many as all the pixels.

As described above, according to the endoscope device of the third modified example, the monochromatic image data by the special light 1 and the monochromatic image data by the special light 2 may be obtained as well as the color image data by carrying out a shooting or recording operation once. The color image data is formed by using the floating gates FG1 and FG2 and the monochromatic image data is formed by using the floating gates FG3. A little time is necessary between the storage of the electric charges for forming the color image data and the storage of the electric charges for forming the monochromatic image data. Therefore, even when the object to be shot or recorded moves, a possibility is low that the object to be shot or recorded may shift between the color image data and the monochromatic image data. As a result, for the same object to be shot or recorded, an image may be observed under different conditions, so that a proper diagnosis may be realized.

There is certain biological information as an object to be observed which may be observed only by applying a plurality of special lights thereto. When the plurality of special lights are supposed to include the special light 1 and the special light 2, in such a case, after the fourth reset operation is completed, the special light 1 and the special light 2 may be emitted at the same time or continuously, in the timing chart shown in FIG. 15 to carry out an exposure operation. The electric charges obtained by the exposure operation may be injected to the floating gates FG3 of all the pixels 100. Then, image pick-up signals may be read from the floating gates FG3 to from image data from the image pick-up signals. In such a way, the light emitting timing of the lights is changed so that various kinds of image data may be obtained and a flexible diagnosis meeting a condition may be realized.

Also in the third modified example, the electric charges corresponding to the R light may be stored in the floating gates half as many as all the floating gates FG2 and the electric charges corresponding to the B light may be stored in the remaining floating gages FG2 half as may as all the floating gates FG2. The electric charges corresponding to the special light 1 may be stored in the floating gates FG3 half as many as all the floating gates FG3 and the electric charges corresponding to the special light 2 may be stored in the remaining floating gages FG3 half as may as all the floating gates FG3. Thus, the floating gates may not be divided into those of the odd number lines and those of the even number lines.

### Fourth Modified Example

In a fourth modified example, will be described the modified examples of the inner structures of the pixel parts 100 of the solid-state image pick-up elements 10 of the endoscope device shown in FIG. 1 or the endoscope devices of the first to third modified examples.

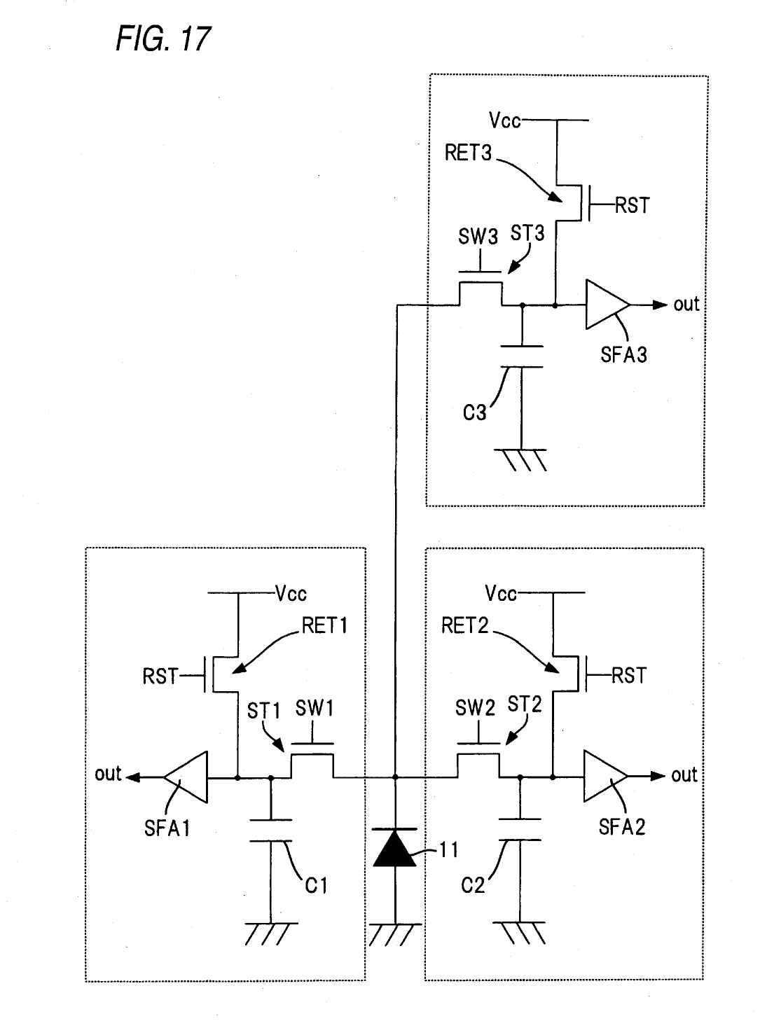

FIG. 17 is a diagram showing the fourth modified example of the endoscope device shown in FIG. 1 and an equivalent circuit diagram showing a modified structural example of the pixel part shown in FIG. 3. The structure shown in FIG. 17 may be applied respectively to the pixel parts of the solid-state image pick-up elements of the endoscope devices described in the first to third modified examples.

In the pixel part shown in FIG. 17, as a plurality of electric charge storage parts that may selectively store electric charges generated in a photoelectric conversion part 11, a floating diffusion capacitance C1, a floating diffusion capacitance C2 and a floating diffusion capacitance C3 are provided. Further, the pixel part shown in FIG. 17 includes a switch transistor ST1, a reset transistor RET1 and a source follower amplifier SFA1 provided correspondingly to the floating diffusion capacitance C1, a switch transistor ST2, a reset transistor RET2 and a source follower amplifier SFA2 provided correspondingly to the floating diffusion capacitance C2, and a switch transistor ST3, a reset transistor RETS and a source follower amplifier SFA3 provided correspondingly to the floating diffusion capacitance C3.

The switch transistor ST1 controls the electric charges in the photoelectric conversion part 11 to be transferred to the floating diffusion capacitance C1. The source follower amplifier SFA1 is connected to the floating diffusion capacitance C1 to output a signal corresponding to an amount of the electric charges transferred to the floating diffusion capacitance C1. The reset transistor RET1 serves to reset the potential of the floating diffusion capacitance C1 to a source voltage Vcc.

The switch transistor ST2 controls the electric charges in the photoelectric conversion part 11 to be transferred to the floating diffusion capacitance C2. The source follower amplifier SFA2 is connected to the floating diffusion capacitance C2 to output a signal corresponding to an amount of the electric charges transferred to the floating diffusion capacitance C2. The reset transistor RET2 serves to reset the potential of the floating diffusion capacitance C2 to a source voltage Vcc.

The switch transistor ST3 controls the electric charges in the photoelectric conversion part 11 to be transferred to the floating diffusion capacitance C3. The source follower amplifier SFA3 is connected to the floating diffusion capacitance C3 to output a signal corresponding to an amount of the electric charges transferred to the floating diffusion capacitance C3. The reset transistor RETS serves to reset the potential of the floating diffusion capacitance C3 to a source voltage Vcc.

In the endoscope device on which the solid-state image pick-up element having the pixel parts shown in FIG. 17, in accordance with an instruction for shooting or recording an object, initially, the switch transistors ST1 and the reset transistors RET1 of all the pixel parts are respectively turned on. Thus, unnecessary electric charges in the photoelectric conversion parts 11 are completely transferred to the floating diffusion capacitances C1 and discharged to the drains of the reset transistors RET1 from them. Then, the switch transistors ST1 and the reset transistors RET1 of all the pixel parts are respectively turned off and an exposure by a G light is started at the same time. When an exposure period is finished, the switch transistors ST1 of all the pixel parts are turned on to completely transfer the electric charges generated in the photoelectric conversion parts 11 to the floating diffusion capacitances C1 and turn off the switch transistors ST1.

Then, the switch transistors ST2 and the reset transistors RET2 of all the pixel parts are respectively turned on. Thus, remaining electric charges in the photoelectric conversion parts 11 are completely transferred to the floating diffusion capacitances C2 and discharged to the drains of the reset transistors RET2 from them. Then, the switch transistors ST2 and the reset transistors RET2 of all the pixel parts are respectively turned off and an exposure by an R light is started at the same time. When an exposure period is finished, the switch transistors ST2 of all the pixel parts are turned on to completely transfer the electric charges generated in the photoelectric conversion parts 11 to the floating diffusion capacitances C2 and turn off the switch transistors ST2.

Then, the switch transistors ST3 and the reset transistors RET3 of all the pixel parts are respectively turned on. Thus, remaining electric charges in the photoelectric conversion parts 11 are completely transferred to the floating diffusion capacitances C3 and discharged to the drains of the reset transistors RET3 from them. Then, the switch transistors ST3 and the reset transistors RET3 of all the pixel parts are respectively turned off and an exposure by a B light is started at the same time. When an exposure period is finished, the switch transistors ST3 of all the pixel parts are turned on to completely transfer the electric charges generated in the photoelectric conversion parts 11 to the floating diffusion capacitances C3 and turn off the switch transistors ST3.

After the storage of the electric charges is finished, a driving operation is carried out, to all lines, for reading image pick-up signals corresponding to the amounts of the electric charges transferred to the floating diffusion capacitances C1 to an external part by the source follower amplifiers SFA1 to read the image pick-up signals. Then, a driving operation is carried out, to all lines, for reading image pick-up signals corresponding to the amounts of the electric charges transferred to the floating diffusion capacitances C2 to an external part by the source follower amplifiers SFA2 to read the image pick-up signals. Then, a driving operation is carried out, to all lines, for reading image pick-up signals corresponding to the amounts of the electric charges transferred to the floating diffusion capacitances C3 to an external part by the source follower amplifiers SFA3 to read the image pick-up signals.

Even in the above-described structure, a heat generation is suppressed and intervals between image shooting or recording operations of three times may be shortened, so that the device may be miniaturized and a diagnostic accuracy may be improved. Further, in the structural example shown in FIG. 17, since the electric charges may be selectively stored in the floating diffusion capacitances respectively, the driving methods described in the first to third modified examples may be employed.

### Fifth Modified Example

In the endoscope device of the second modified example, in order to form the color image data, the two electric charge storage parts including the floating gates FG1 and the electric charge storage parts including the floating gates FG2 may be adequately included. Thus, in the structure of an endoscope device of a fifth modified example, the number of electric charge storage parts in a pixel part 100 of a solid-state image pick-up element 10 is set to two.

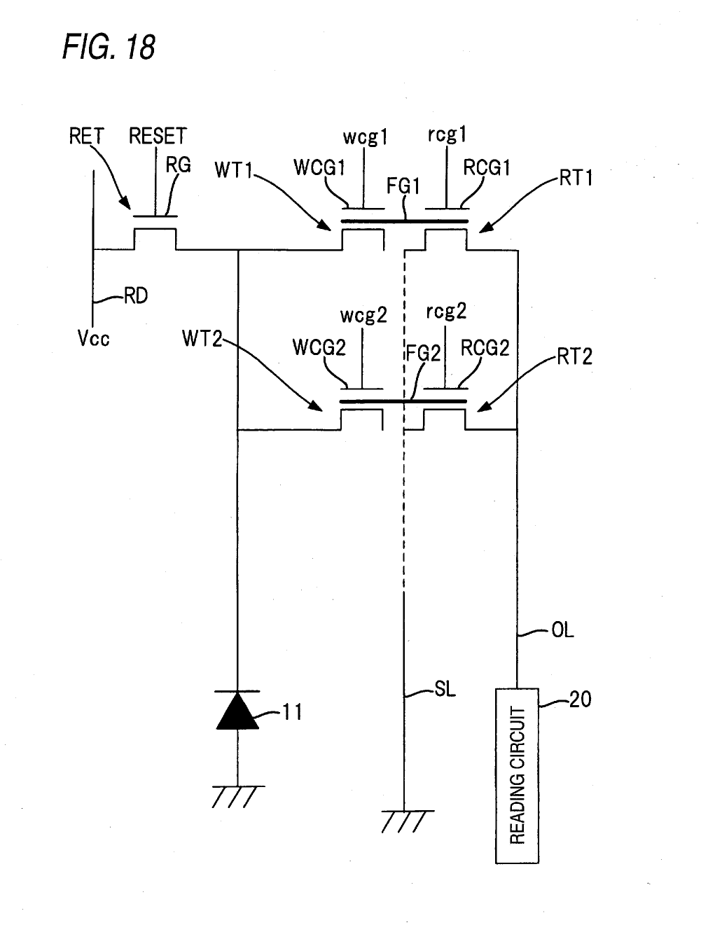

FIG. 18 is a diagram showing the fifth modified example of the endoscope device shown in FIG. 1 and an equivalent circuit diagram showing a modified structural example of the pixel part shown in FIG. 3. The pixel part shown in FIG. 18 has a structure in which the third electric charge storage part (the writing transistor WT3, the reading transistor RT3) of the pixel part shown in FIG. 3 is deleted.

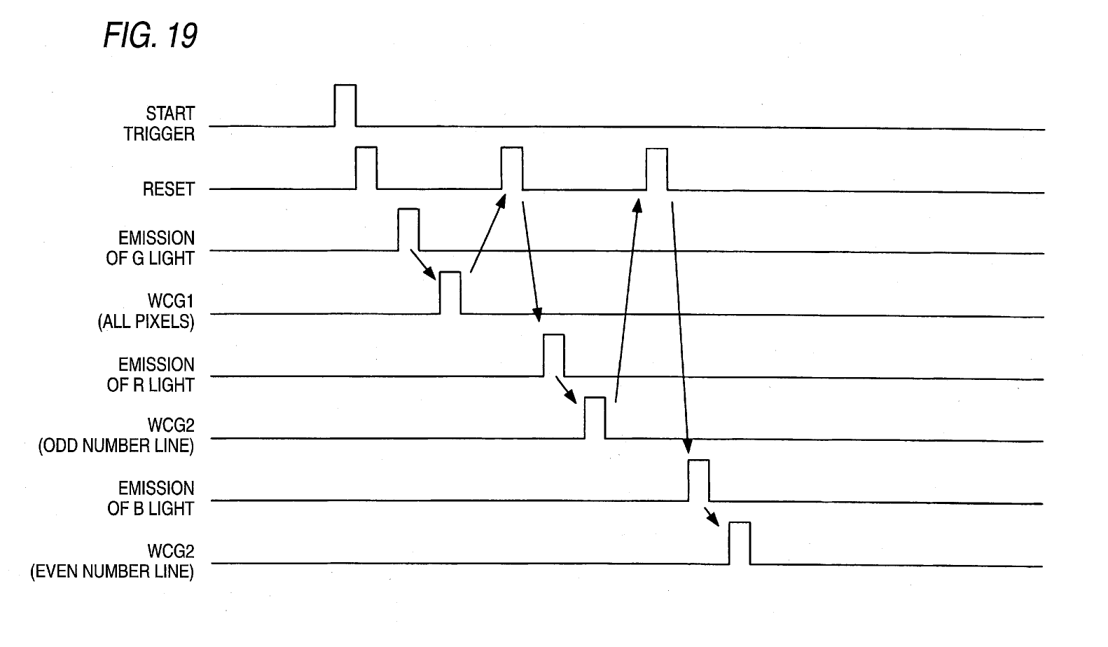

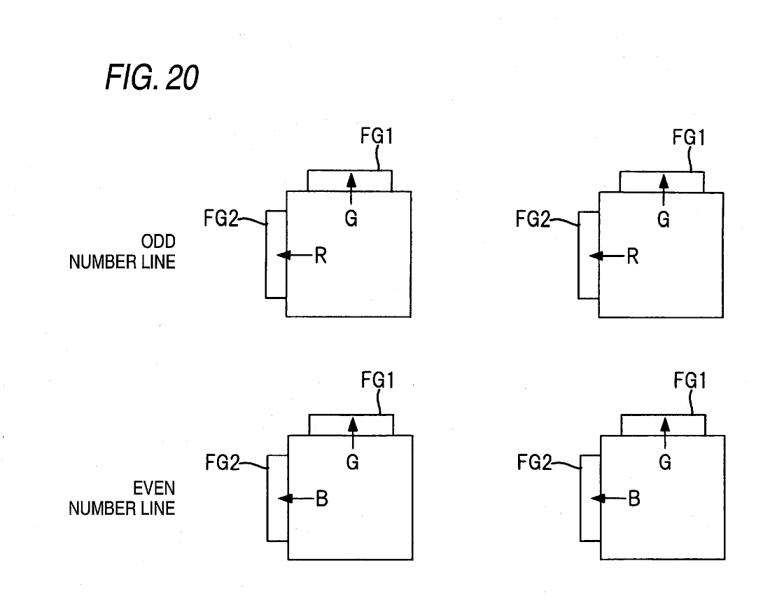

FIG. 19 is a timing chart for explaining the operation of the endoscope device of the fifth modified example. FIG. 20 is a schematic view for explaining the operation of the endoscope device of the second modified example. In FIG. 20, four pixel parts of 2 rows×2 columns in total are schematically shown.

When an operating part 25 is operated to instruct an object to be shot or recorded, this instruction is inputted to a system control part 24 and the system control part 24 informs the solid-state image pick-up element 10 of the instruction for shooting or recording the object.

When the solid-state image pick-up element 10 receives the instruction for shooting or recording the object, a control part 40 considers it as a start trigger to supply reset pulses to reset gates RG of reset transistors RET of all pixel parts 100. Thus, unnecessary electric charges respectively stored in photoelectric conversion parts 11 of the pixel parts 100 are discharged to the drains of the reset transistors RET.

After a reset operation is completed, the system control part 24 outputs an instruction to a light source driving part 21 to emit a G light from an LED 1b. In FIG. 19, the G light is emitted after a little time when the reset pulses are supplied, however, the G light may be emitted at the same time as the completion of the reset operation.

The G light is emitted, for instance, only during an exposure period set by the endoscope device. During the emission of the G light, in the pixel parts 100 of the solid-state image pick-up element 10 respectively, lights incident from the object are incident on the photoelectric conversion parts 11 and the electric charges corresponding to the G light are generated and stored therein.

After the exposure period is finished, the control part 40 supplies writing pulses to the writing control gates WCG1 of all the pixel parts 100 to store the electric charges generated in the photoelectric conversion parts 11 during the exposure period in floating gates FG1. In supplying the writing pulses, either a method for starting the supply of the writing pulses at the same time as the completion of the exposure period or a method for starting the supply of the writing pulses at the same time as the start of the exposure period and completing the supply of the writing pulses at the same time as the completion of the exposure period may be employed.

In accordance with the supply of the writing pulses, as shown in FIG. 20, the electric charges (the electric charges by the G light, shown by “G” in the drawing) generated in the pixel parts 100 are stored respectively in the floating gates FG1 of the pixel parts 100.

When the storage of the electric charges in the floating gates FG1 is finished, the control part 40 supplies again reset pulses to the reset gates RG of the reset transistors RET of all the pixel parts 100. Thus, remaining electric charges which are hardly injected to the floating gates FG1 from the photoelectric conversion parts 11 to be left are discharged to the drains of the reset transistors RET.

After a second reset operation is completed, the system control part 24 outputs an instruction to the light source driving part 21 to emit an R light from an LED 1a. In FIG. 19, the R light is emitted after a little time when the reset pulses are supplied, however, the R light may be emitted at the same time as the completion of the reset operation.

The R light is emitted, for instance, only during an exposure period set by the endoscope device. During the emission of the R light, in the pixel parts 100 of the solid-state image pick-up element 10 respectively, lights incident from the object are incident on the photoelectric conversion parts 11 and the electric charges corresponding to the R light are generated and stored therein.

After the exposure period is finished, the control part 40 supplies writing pulses to the writing control gates WCG2 of the pixel parts 100 of odd number lines to store the electric charges generated in the photoelectric conversion parts 11 during the exposure period in floating gates FG2. In supplying the writing pulses, either a method for starting the supply of the writing pulses at the same time as the completion of the exposure period or a method for starting the supply of the writing pulses at the same time as the start of the exposure period and completing the supply of the writing pulses at the same time as the completion of the exposure period may be employed.

In accordance with the supply of the writing pulses, as shown in FIG. 20, the electric charges (the electric charges by the R light, shown by “R” in the drawing) generated in the pixel parts 100 are stored respectively only in the floating gates FG2 of the pixel parts 100 of the odd number lines.

When the storage of the electric charges in the floating gates FG2 of the pixel parts 100 of the odd number lines is finished, the control part 40 supplies again reset pulses to the reset gates RG of the reset transistors RET of all the pixel parts 100. Thus, remaining electric charges which are hardly injected to the floating gates FG2 from the photoelectric conversion parts 11 to be left are discharged to the drains of the reset transistors RET.

After a third reset operation is completed, the system control part 24 outputs an instruction to the light source driving part 21 to emit a B light from an LED 1c. In FIG. 19, the B light is emitted after a little time when the reset pulses are supplied, however, the B light may be emitted at the same time as the completion of the reset operation.

The B light is emitted, for instance, only during an exposure period set by the endoscope device. During the emission of the B light, in the pixel parts 100 of the solid-state image pick-up element 10 respectively, lights incident from the object are incident on the photoelectric conversion parts 11 and the electric charges corresponding to the B light are generated and stored therein.

After the exposure period is finished, the control part 40 supplies writing pulses to the writing control gates WCG2 of the pixel parts 100 of even number lines to store the electric charges generated in the photoelectric conversion parts 11 during the exposure period in floating gates FG2. In supplying the writing pulses, either a method for starting the supply of the writing pulses at the same time as the completion of the exposure period or a method for starting the supply of the writing pulses at the same time as the start of the exposure period and completing the supply of the writing pulses at the same time as the completion of the exposure period may be employed.

In accordance with the supply of the writing pulses, as shown in FIG. 20, the electric charges (the electric charges by the B light, shown by “B” in the drawing) generated in the pixel parts 100 are stored respectively only in the floating gates FG2 of the pixel parts 100 of the even number lines.

After the electric charges are completely stored in the floating gates FG2, the control part 40 pre-charges the drains of the reading transistors RT1 of the pixel parts 100 respectively in a first line to begin to apply a lamp wave form voltage to the reading control gates RCG1 of the pixel parts 100 of the first line. Then, count values corresponding to the value of the lamp wave form voltage when the drain potentials of the reading transistors RT1 of the first line drop are respectively held in reading circuits 20 and the count values are outputted from an output amplifier 60 as image pick-up signals. The control part 40 carries out a similar driving operation in lines after a second line to output first image pick-up signals (G signals) corresponding to the electric charges stored in the floating gates FG1 of all lines.

Then, the control part 40 pre-charges the drains of the reading transistors RT2 of the pixel parts 100 respectively in the first line to begin to apply the lamp wave form voltage to the reading control gates RCG2 of the pixel parts 100 of the first line. Then, count values corresponding to the value of the lamp wave form voltage when the drain potentials of the reading transistors RT2 of the first line drop are respectively held in the reading circuits 20 and the count values are outputted from the output amplifier 60 as image pick-up signals. The control part 40 carries out a similar driving operation in lines after a second line to output second image pick-up signals (R signals and B signals) corresponding to the electric charges stored in the floating gates FG2 of all lines.

After the second image pick-up signals are outputted, the control part 40 sets the potentials of the writing control gates WCG1 and WCG2 and the reading control gates RCG1 and RCG2 of all the pixel parts 100 to—Vcc and the potential of a semiconductor substrate to Vcc. Thus, the electric charges stored in the floating gates FG1 and FG2 are drawn out to the semiconductor substrate and erased.

The above-described operations are carried out within one frame period.

A signal processing part 23 forms color image data by the G signals, the R signals or the B signals respectively outputted from the pixel parts 100 of the solid-state image pick-up element 10. Specifically, the R signals or the B signals that are not obtained from the pixel parts 100 are interpolated by using R signals and B signals obtained from the pixel parts 100 in the periphery of the pixel parts 100 to form the R signal, the G signal and the B signal for one pixel part 100. A luminance signal and a color difference signal are formed from these signals to form the color image data.

As described above, according to the endoscope device of the fifth modified example, the color image data whose color divergence is suppressed may be formed only by providing the two electric charge storage parts respectively in the pixel parts 100 of the solid-state image pick-up element 10. Accordingly, the size of the pixel part may be more reduced and the photoelectric conversion part may be more enlarged than a case that the three electric charge storage parts are respectively provided. Thus, a multi-pixels and a high sensitivity may be realized.

Also in the fifth modified example, the electric charges corresponding to the R light may be stored in the floating gates FG2 half as many as all the floating gates FG2, and the electric charges corresponding to the B light may be stored in remaining floating gates FG2 half as many as all the floating gates FG2. Thus, the floating gates FG2 may not be divided into the floating gates FG2 of the odd number lines and the floating gates FG2 of the even number lines.

In the above description, a light source 1 is formed with the LEDs for emitting the lights respectively having different wavelengths. However, the lights of the respectively different wavelengths may be emitted by a white light source and a spectral filter inserted into a front surface thereof. In this case, when the plurality of lights is emitted at the same time, the above-described structure may not be employed.

### Sixth Modified Example

In this modified example, an example will be described in which the two electric charge storage parts included in the pixel parts 100 of the solid-state image pick-up element described in the fifth modified example are respectively formed with one transistors.

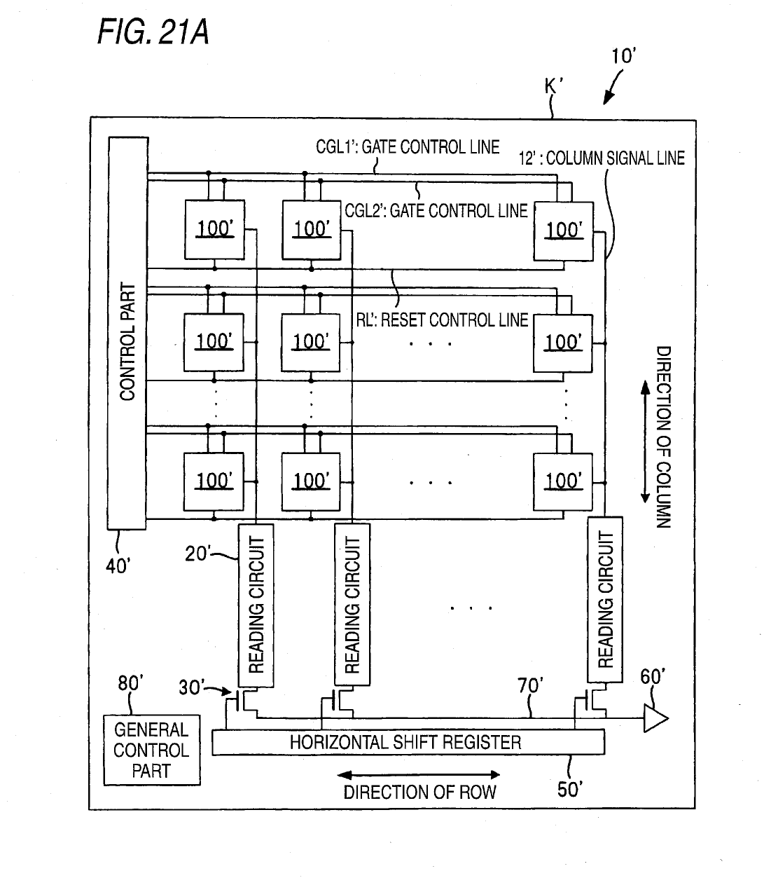

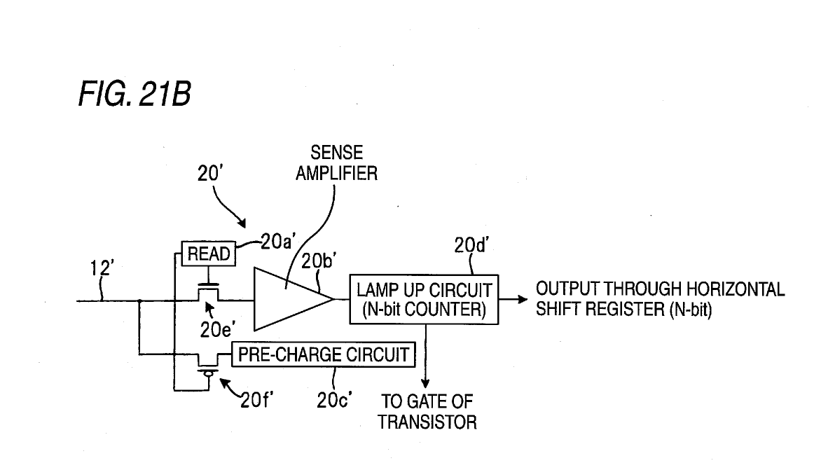

FIGS. 21A and 21B are a schematic plan view showing a schematic structure of another example of the solid-state image pick-up element for explaining the one exemplary embodiment of the present invention. FIG. 21A is a diagram showing an entire part of the solid-state image pick-up element and FIG. 21B is a diagram showing a structural example of a reading circuit of the solid-state image pick-up element in FIG. 21A. The image pick-up element 10′ shown in FIG. 21 includes pixel part 100′, reading circuits 20′, an output circuit (transistors 30′, a signal line 70′, a horizontal shift register 50′, an output part 60′), a control part 40′ and a general control part 80′.

A plurality of pixel parts 100′ are arranged in a two-dimensional form (in this example, a square grid form) in the directions of columns and the directions of rows orthogonal therewith on a semiconductor substrate K′.

The reading circuit 20′ is provided for each pixel column including the pixel parts 100′ arranged in the direction of the column to read image pick-up signals from the pixel parts 100′ respectively.

The output circuit serves to output image pick-up signals of one pixel row read by the reading circuits 20′.

The control part 40′ controls the pixel parts 100′ respectively.

The general control part 80′ serves to generally control the entire part of the solid-state image pick-up element 10′. The solid-state image pick-up element 10′ is operated under the control of parts by the general control part 80′ in accordance with a control from a system control part of an image pick-up device on which the image pick-up element is mounted.

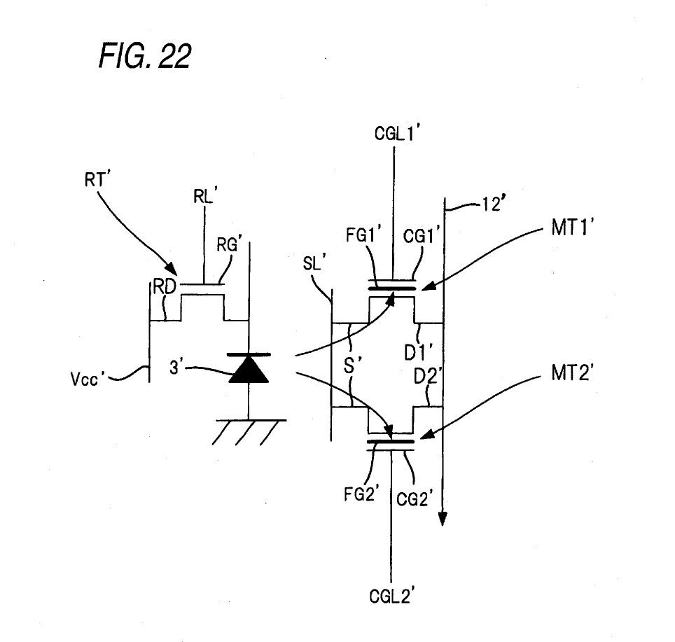

FIG. 22 is a diagram showing an equivalent circuit of the pixel part in the solid-state image pick-up element shown in FIG. 21. As shown in FIG. 22, the pixel part 100′ includes a photoelectric conversion part 3′, a nonvolatile memory transistor MT1′, a nonvolatile memory transistor MT2′ and a reset transistor RT′.

The photoelectric conversion part 3′ is formed in the semiconductor substrate K′. The nonvolatile memory transistor MT1′ has an MOS transistor structure including a floating gate FG1′ as an electric charge storage area formed in an upper part of the semiconductor substrate K′ and a control gate CG1′ as a gate electrode. The nonvolatile memory transistor MT2′ has an MOS transistor structure including a floating gate FG2′ as an electric charge storage area formed in an upper part of the semiconductor substrate K′ and a control gate CG2′ as a gate electrode. The reset transistor RT′ serves to reset electric charges in the photoelectric conversion part 3′. The nonvolatile memory transistor MT1′ and the nonvolatile memory transistor MT2′ respectively function as electric charge storage parts that may selectively store the electric charges generated in the photoelectric conversion part 3′.

The outputs (drain areas D1′, D2′) of the nonvolatile memory transistor MT1′ and the nonvolatile memory transistor MT2′ are respectively commonly connected to a column signal line 12′ as a signal output line provided for each pixel column and the reading circuit 20′ is connected to the column signal line 12′. Source areas S′ of the nonvolatile memory transistors MT1′ and MT2′ are commonly connected to a source line SL′ provided for each pixel column.

The reset transistor RT′ has an MOS structure including a reset drain RD′, the photoelectric conversion part 3′ functioning as a source area and a reset gate RG′ as a gate electrode. To the reset drain RD′, a reset power line Vcc′ for supplying a reset voltage is connected.

To the control gate CG1′ of the nonvolatile memory transistor MT1′, a gate control line CGL1′ provided for each line composed of the pixel parts 100′ arranged in the direction of a row is connected. The gate control line CGL1′ of each line is connected to the control part 40′, so that a voltage may be independently applied for each line.

To the control gate CG2′ of the nonvolatile memory transistor MT2′, a gate control line CGL2′ provided for each line is connected. The gate control line CGL2′ of each line is connected to the control part 40′, so that a voltage may be independently applied for each line.

To the reset gate RG′ of the reset transistor RT′ a reset control line RL′ provided for each line is connected. The reset control line RL′ of each line is connected to the control part 40′, so that a voltage may be independently applied for each line. A reset pulse is applied through the reset control line REL′ from the control part 40′ to turn on the reset transistor RT′ and discharge the electric charges stored in the photoelectric conversion part 3′ to the drain RD′ of the reset transistor RT′.

As shown in FIG. 21B, the reading circuit 20′ includes a reading control part 20a′, a sense amplifier 20b′, a pre-charge circuit 20c′, a lamp up circuit 20d′ and transistors 20e′ and 20f′.

The reading control part 20a′ controls turning on and off of the transistors 20e′ and 20f′. The pre-charge circuit 20c′ serves to supply a prescribed voltage to the column signal line 12′ and pre-charge the column signal line 12′. The sense amplifier 20b′ monitors the voltage of column signal line 12′ to detect the change of the voltage and inform the lamp up circuit 20d′ of the change of the voltage. For instance, the sense amplifier 20b detects that a drain voltage pre-charged by the pre-charge circuit 20c′ drops to invert an output of the sense amplifier.

The lamp up circuit 20d′ incorporates an N-bit counter (for instance, N=about 8 to 12) to supply a lamp wave form voltage that gradually increases or gradually decreases to the control gates CG1′ and CG2′ of the pixel part 100′ through the control part 40′ and output a count value (N combinations of 1 and 0) corresponding to the value of the lamp wave form voltage.

When the voltage of the control gate CG1′ exceeds the threshold voltage of the nonvolatile memory transistor MT1′ under a state that the column signal line 12′ is pre-charged, the nonvolatile memory transistor MT1′ is electrically conducted. At this time, the potential of the pre-charged column signal line 12′ drops. This drop is detected by the sense amplifier 20b′ and an inversion signal is outputted. The lamp up circuit 20d′ holds (latches) the count value corresponding to the value of the lamp wave form voltage when the lamp up circuit 20d′ receives the inversion signal. Thus, the variation(a variation obtained when the threshold voltage is set as a reference under a state that the electric charge is not stored in the floating gate FG1′) of the threshold voltage of the nonvolatile memory transistor MT1′ as a digital value (a combination of 1 and 0) may be read as a signal.

When the voltage of the control gate CG2′ exceeds the threshold voltage of the nonvolatile memory transistor MT2′ under a state that the column signal line 12′ is pre-charged, the nonvolatile memory transistor MT2′ is electrically conducted. At this time, the potential of the pre-charged column signal line 12′ drops. This drop is detected by the sense amplifier 20b′ and an inversion signal is outputted. The lamp up circuit 20d′ holds (latches) the count value corresponding to the value of the lamp wave form voltage when the lamp up circuit 20d′ receives the inversion signal. Thus, the variation (a variation obtained when the threshold voltage is set as a reference under a state that the electric charge is not stored in the floating gate FG2′) of the threshold voltage of the nonvolatile memory transistor MT2′ as a digital value may be read as a signal.

When one horizontal selecting transistor 30′ is selected by the horizontal shift register 50, the counter value held by the lamp up circuit 20d′ connected to the horizontal selecting transistor 30′ is outputted to the signal line 70′ and outputted from the output amplifier 60′ as the image pick-up signal.

A method for reading the change of the threshold voltage of the nonvolatile memory transistors MT1′ and MT2′ as the signals is not limited to the above-described method. For instance, a drain current of the nonvolatile memory transistor MT1′ when a prescribed voltage is applied to the control gate CG1′ and the drain area D1′, and a drain current of the nonvolatile memory transistor MT2′ when a prescribed voltage is applied to the control gate CG2′ and the drain area D2′ may be read as signals.

The control part 40′ controls the nonvolatile memory transistors MT1′ and MT2′ to be driven so as to inject and store the electric charges generated in the photoelectric conversion part 3′ in the floating gates FG1′ and FG2′. In the nonvolatile memory transistor MT1′ (MT2′), a writing pulse is applied to the control gate CG1′ (CG2′) to inject and store the electric charges generated in the photoelectric conversion part 3′ to the floating gate FG1′ (FG2′) by an FN tunnel injection for injecting the electric charges by using a Fowler-Nordheim (F-N) tunnel current, a direct tunnel injection, a hot electron injection or the like.

Further, the control part 40′ carries out a reset driving for discharging outside the electric charges generated and stored in the photoelectric conversion parts 3′ of the pixel parts 100′ respectively to empty the photoelectric conversion parts 3′ and an electric charge erase driving for discharging the electric charges stored in the floating gages FG1′ and FG2′ to the semiconductor substrate to erase the electric charges.

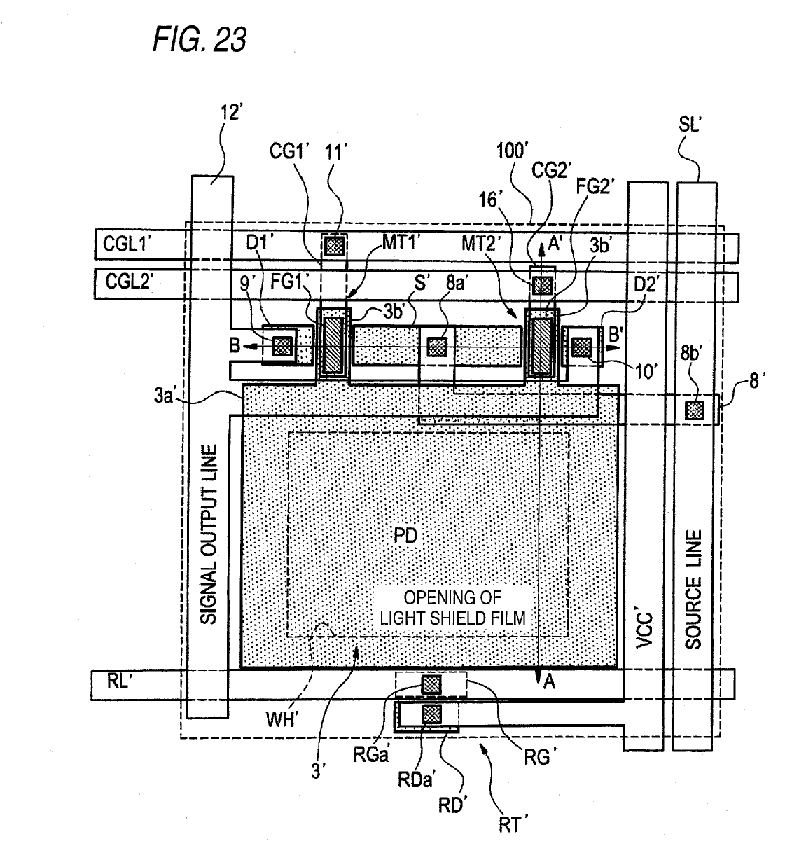

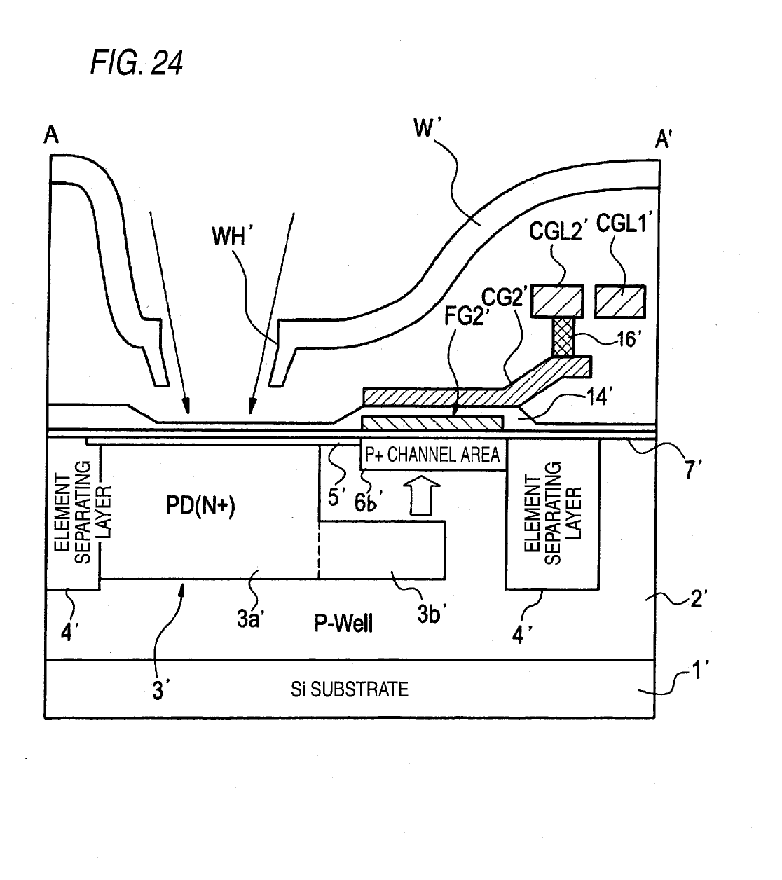

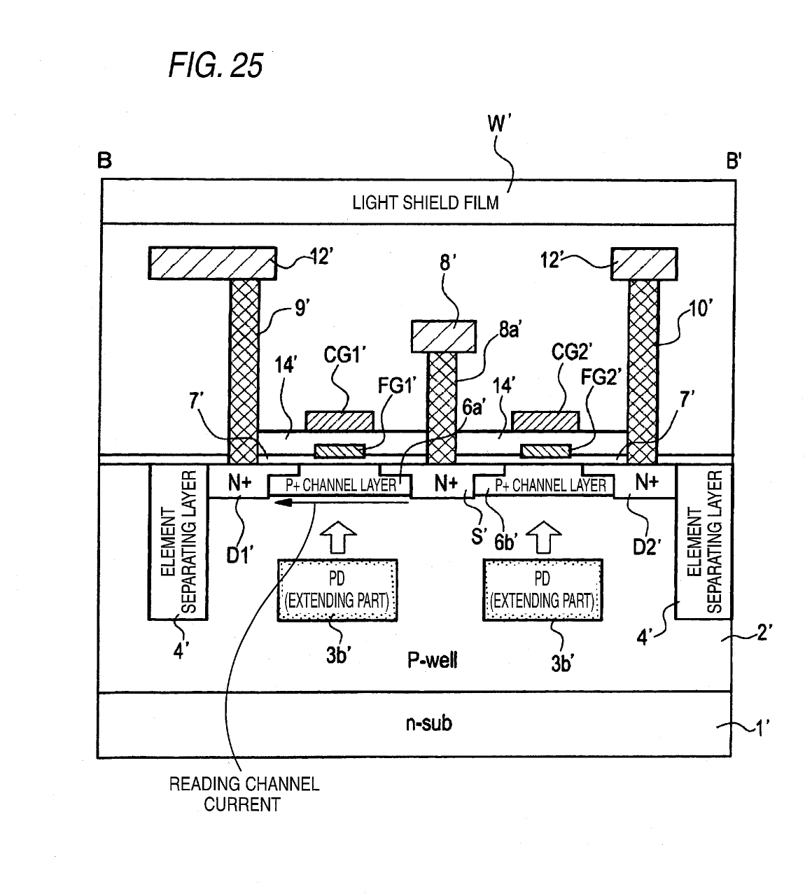

FIG. 23 is a schematic plan view showing a plane layout example of the pixel part of the solid-state image pick-up element shown in FIG. 21. FIG. 24 is a schematic sectional view taken along a line A-A′ of the pixel part shown in FIG. 23. FIG. 25 is a schematic sectional view taken along a line B-B′ of the pixel part shown in FIG. 23.

As shown in FIG. 24, the photoelectric conversion part 3′ is an N type impurity area formed in a P well layer 2′ on an N type silicon substrate 1′ and realizes a photoelectric conversion function by a PN junction of the N type impurity area and the P well layer 2′. The photoelectric conversion part 3′ is what is called an embedded photodiode having a P type impurity layer 5′ formed on its surface to suppress a complete depletion of a dark current. The semiconductor substrate K′ is formed by the N type silicon substrate 1′ and the P well layer 2′.

The adjacent pixel parts 100′ are separated from each other by an element separating layer 4′ formed in the P well layer 2′. To an element separating method, a LOCOS (Local Oxidation of Silicon) method, an STI (Shallow Trench Isolation) method and a method of a high concentration impurity ion injection or the like may be applied.

The source area S′ of the nonvolatile memory transistor MT1′ is an N type impurity area provided adjacently to the photoelectric conversion part 3′ so as to be separated in the direction of a column. Further, the drain area D1′ of the nonvolatile memory transistor MT1′ is an N type impurity area provided adjacently to the source area S′ so as to be separated in the direction of a row. Between the source area S′ and the drain area D1′, a channel area 6a′ as a P type impurity area is formed. The floating gate FG1′ is provided on an upper part of the semiconductor substrate between the source area S′ and the drain area D1′ through an insulating film 7′. In an upper part of the floating gate FG1′, the control gate CG1′ is formed through an insulating film 14′. The channel area 6a′ is an area to which a carrier is supplied in accordance with a voltage applied to the control gate CG1′. Here, P type impurities are injected to an area sandwiched by the source area S′ and the drain area D1′ to form the channel area 6a′, however, the area may remain to be the P well layer 2′ as it is.

The drain area D2′ of the nonvolatile memory transistor MT2′ is an N type impurity area provided adjacently to the source area S′ so as to be separated in the direction of a row. Between the source area S′ and the drain area D2′, a channel area 6b′ as a P type impurity area is formed. The floating gate FG2′ is provided on an upper part of the semiconductor substrate between the source area S′ and the drain area D2′ through an insulating film 7′. In an upper part of the floating gate FG2′, the control gate CG2′ is formed through an insulating film 14′. The channel area 6b′ is an area to which a carrier is supplied in accordance with a voltage applied to the control gate CG2′. Here, P type impurities are injected to an area sandwiched by the source area S′ and the drain area D2′ to form the channel area 6b′, however, the area may remain to be the P well layer 2′ as it is.

As an electrically conductive material forming the control gates CG1′ and CG2′, for instance, poly-silicon may be employed. Doped poly-silicon doped with phosphorus (P), arsenic (As) and boron (B) of high concentration may be employed. Otherwise, Silicide or Salicide (Self-align Silicide) may be employed which is obtained by combining various kinds of metals such as titanium (Ti) or tungsten (W) with silicon. As an electrically conductive material forming the floating gates FG1′ and FG2′, the same materials of the control gates CG1′ and CG2′ may be employed.

In the layout example shown in FIG. 23, the source area S′ and the drain areas D1′ and D2′ are arranged in parallel in the direction of the row and the floating gates FG1′ and FG2′ and the control gates CG1′ and CG2′ are formed in elongated shapes between the source area and the drain areas so as to be extended in the directions of columns. The control gate CG1′ is extended to a lower part of the gate control line CGL1′ as an aluminum wiring extending in the direction of a row and connected therein to the gate control line CGL1′ by a contact part 11′ made of aluminum.

The control gate CG2′ is extended to a lower part of the gate control line CGL2′ as an aluminum wiring extending in the direction of a row and connected therein to the gate control line CGL2′ by a contact part 16′ made of aluminum.

In an upper part of the drain areas D1′ and D2′, a part of the column signal line 12′ as an aluminum wiring extending in the direction of a column is extended, and the part is electrically connected to the drain area D1′ by a contact part 9′ made of aluminum and the part is electrically connected to the drain area D2′ by a contact part 10′ made of aluminum.

On the source area S′, a contact part 8a′ made of aluminum is formed and a wiring 8′ is connected to the contact part 8a′. The wiring 8′ passes a lower part of a reset power line Vcc′ as an aluminum wiring extending in the direction of a column and is extended to a lower part of a source line SL′. The wiring 8′ is electrically connected to the source line SL′ by a contact part 8b′ made of aluminum. The source line SL′ is provided for each column composed of the pixel parts 100′ arranged in the direction of the column and connected to a prescribed potential (for instance, a ground potential).

The reset transistor RT′ has the MOS transistor structure including the photoelectric conversion part 3′ functioning as the source area, a drain area RD′ as an N type impurity area provided adjacently to the photoelectric conversion part 3′ so as to be separated from the photoelectric conversion part 3′ in the direction of a column and the reset gate RG′ provided in an upper part of the semiconductor substrate between the photoelectric conversion part 3′ and the drain area RD′ through an insulating film 7′.

In the layout example shown in FIG. 23, the reset gate RG′ is arranged in a lower part of the reset control line RL′ as an aluminum wiring which extends in the direction of a row and connected therein to the reset control line RL′ by a contact part RGa′ made of aluminum.

In an upper part of the drain area RD′, a part of the reset power line Vcc′ is extended and the part is electrically connected to the drain area RD′ by a contact part RDa′ made of aluminum. The reset power line Vcc′ is provided for each column composed of the pixel parts 100′ arranged in the direction of the column and connected to a prescribed source voltage.

The arrangement of the reset transistor RT′ or the nonvolatile memory transistors MT1′ and MT2′ is not limited to that shown in FIG. 23 and these transistors may be suitably arranged depending on a space.

In the positional relation of various kinds of wirings, the source line SL′, the reset power line Vcc′ and the column signal line 12′ are formed in the upper layer of a layer of the gate control lines CGL1′ and CGL2′, the reset control line RL′ and the wiring 8′.

In the structure of the pixel part 100′, a light is not allowed to be incident on other area than a part of the photoelectric conversion part 3′ by a light shield film W′ formed with, for instance, tungsten. As shown in FIG. 24 and FIG. 25, in the upper part of the semiconductor substrate (the upper parts of the source line SL′, the reset power line Vcc′ and the column signal line 12′), the light shield film W′ is formed which has an opening WH′ formed in the upper part of a part of the photoelectric conversion part 3′.

In the solid-state image pick-up element 10′, for the purpose of improving an electric charge injection efficiency to the floating gates FG1′ and FG2′, as shown in FIG. 24 and FIG. 25, the photoelectric conversion part 3′ is extended not only to a lower part of the opening WH′ of the light shield film W′, but also to lower parts of the channel areas 6a′ and 6b′ of the nonvolatile memory transistors MT1′ and MT2′.

As shown in FIGS. 24 and 25, the photoelectric conversion part 3′ includes a main body part 3a′ formed in the lower part of the opening WH′ and an extending part 3b′ extending to the lower part of the channel area 6a′ (6b′) from the main body part 3a′. In FIG. 24, a boundary line (a broken line) is shown between the main body part 3a′ and the extending part 3b′, however, the boundary line is provided for explanation. Actually, the boundary line does not exist.

The main body part 3a′ is formed in the lower part of the opening WH′ to receive lights. The extending parts 3b′ are extended to the lower parts of the channel areas 6a′ and 6b′ of the nonvolatile memory transistors MT1′ and MT2′ in the P well layer 2′ from the main body part 3a′. The extending parts 3b′ are formed and extended, as shown in a plan view, from positions of the main body part 3a′ opposed to areas between the source area S′ and the drain areas D1′ and D2′ to the areas in the directions of columns. Namely, in a plan view, in the area where the nonvolatile memory transistors MT1′ and MT2′ or the reset transistor RT′ are formed, the photoelectric conversion part 3′ is provided so that the photoelectric conversion part 3′ exists only in the lower parts of the channel areas 6a′ and 6b′ of the nonvolatile memory transistors MT1′ and MT2′. The extending parts 3b′ may be formed so as to be extended not only to the lower parts of the channel areas 6a′ and 6b′, but also to the lower parts of the entire parts of the nonvolatile memory transistors MT1′ and MT2′.

The channel area 6a′ (6b′) is provided immediately below the control gate CG1′ (CG2′) and the floating gate FG1′ (FG2′). Accordingly, the photoelectric conversion part 3′ is extended to the lower part of the channel area 6a′ (6b′) (preferably, all of a range overlapped on the channel area 6a′ (6b′)in a plan view, so that when the electric charges in the photoelectric conversion part 3′ are injected to the floating gate FG1′ (FG2′) by the FN tunnel injection or the direct tunnel injection, an electric field may be substantially vertically applied to the floating gate (FG1′ (FG2′) from the photoelectric conversion part 3′ by a voltage (CG voltage) applied to the control gate CG1′ (CG2′). Thus, the electric charges in the photoelectric conversion part 3′ are liable to be accelerated toward the control gate CG1′ (CG2′). As a result, a tunneling may be generated by a low CG voltage.

In the solid-state image pick-up element 10′, since the channel area 6a′ (6b′) is ensured and the photoelectric conversion part 3′ is extended to the lower part of the channel area 6a′ (6b′) at the same time, the size of the overlapped part of the photoelectric conversion part 3′ and the control gate CG1′ (CG2′) is not restricted. Thus, an electric field direction may be made to be substantially vertical. As a result, a tunnel current may be efficiently generated.

The photoelectric conversion part 3′ may control a length parallel to the surface of the substrate by controlling a mask pattern during an injection of ions and may control a length vertical to the surface of the substrate by controlling ion injection energy. In such a way, the photoelectric conversion part 3′ may be formed that includes the main body part 3a′ and the extending part 3b′.

An operation of the endoscope device shown in FIG. 1 on which the solid-state image pick-up element 10′ shown in FIG. 21 is mounted will be described below.

When the solid-state image pick-up element 10′ receives an instruction for shooting or recording an object, the control part 40′ considers it as a start trigger to supply reset pulses to the reset gates RG′ of the reset transistors RT′ of all the pixel parts 100′. Thus, unnecessary electric charges respectively stored in the photoelectric conversion parts 3′ of the pixel parts 100′ are discharged to the drains of the reset transistors RT∝.

After the reset operation is completed, a system control part 24 outputs an instruction to a light source driving part 21 to emit a G light from an LED 1b. The G light is emitted, for instance, only during an exposure period set by the endoscope device. During the emission of the G light, in the pixel parts 100′ of the solid-state image pick-up element 10′ respectively, lights incident from the object are incident on the photoelectric conversion parts 3′ and the electric charges corresponding to the G light are generated and stored.

After the exposure period is finished, the control part 40′ supplies writing pulses to the control gates CG1′ of all the pixel parts 100′ to store the electric charges generated in the photoelectric conversion parts 3′ during the exposure period in the floating gates FG1′.

In accordance with the supply of the writing pulses, the electric charges generated in the pixel parts 100′ are stored respectively in the floating gates FG1′ of the pixel parts 100′.

When the storage of the electric charges in the floating gates FG1′ is finished, the control part 40′ resets again the photoelectric conversion parts 3′ of all the pixel parts 100′.

After the second reset operation is completed, the system control part 24 outputs an instruction to the light source driving part 21 to emit an R light from an LED 1a. The R light is emitted, for instance, only during an exposure period set by the endoscope device. During the emission of the R light, in the pixel parts 100′ of the solid-state image pick-up element 10′ respectively, lights incident from the object are incident on the photoelectric conversion parts 3′ and the electric charges corresponding to the R light are generated and stored.

After the exposure period is finished, the control part 40′ supplies writing pulses to the control gates CG2′ of the pixel parts 100′ of odd number lines to store the electric charges generated in the photoelectric conversion parts 3′ during the exposure period in the floating gates FG2′.

In accordance with the supply of the writing pulses, the electric charges generated in the pixel parts 100′ are stored only in the floating gates FG2′ of the pixel parts 100′ of the odd number lines.

When the storage of the electric charges in the floating gates FG2′ of the pixel parts 100′ of the odd number lines is finished, the control part 40′ resets again the photoelectric conversion parts 3′ of all the pixel parts 100′.

After the third reset operation is completed, the system control part 24 outputs an instruction to the light source driving part 21 to emit a B light from an LED 1c. The B light is emitted, for instance, only during an exposure period set by the endoscope device. During the emission of the B light, in the pixel parts 100′ of the solid-state image pick-up element 10′ respectively, lights incident from the object are incident on the photoelectric conversion parts 3′ and the electric charges corresponding to the B light are generated and stored therein.

After the exposure period is finished, the control part 40′ supplies writing pulses to the control gates CG2′ of the pixel parts 100′ of even number lines to store the electric charges generated in the photoelectric conversion parts 3′ during the exposure period in the floating gates FG2′.

In accordance with the supply of the writing pulses, the electric charges (the electric charges by the B light) generated in the pixel parts 100′ are stored respectively only in the floating gates FG2′ of the pixel parts 100′ of the even number lines.

After the electric charges are completely stored in the floating gates FG2′, the reading control part 20a′ turns on the transistor 20f′ to pre-charge the column signal line 12′. Then, the reading control part 20a′ turns on the transistor 20e′ to electrically conduct the column signal line 12′ to the sense amplifier 20b′. Under this state, the lamp up circuit 20d′ begins, through the control part 40′, to apply a lamp wave form voltage (a Vth reading voltage) to the control gates CG1′ of the pixel parts 100′ of a first line (count values after the start of application of the lamp wave form voltage are up counted from, for instance, an initial value (for instance, zero). After the lamp wave form voltage is applied, when the drain potentials of the nonvolatile memory transistors MT1′ of the pixel parts 100′ of the first line drop, the count values corresponding to the value of the lamp wave form voltage at that time are respectively held in the reading circuits 20′. The held count values are outputted from the output amplifier 60′ through the signal line 70′ under the control of the horizontal shift register 50′. After the count values are outputted, the transistor 20f′ is turned off to stop the application of the lamp wave form voltage and reset the count values. A similar driving operation is carried out after a second line to output first image pick-up signals (G signals) corresponding to the electric charges stored in the floating gates FG1′ of all lines.

Then, the reading control part 20a′ turns on the transistor 20f′ to pre-charge the column signal line 12′. Then, the reading control part 20a′ turns on the transistor 20e′ to electrically conduct the column signal line 12′ to the sense amplifier 20b′. Under this state, the lamp up circuit 20d′ begins, through the control part 40′, to apply a lamp wave form voltage (a Vth reading voltage) to the control gates CG2′ of the pixel parts 100′ of a first line (count values after the start of application of the lamp wave form voltage are up counted from, for instance, an initial value (for instance, zero). After the lamp wave form voltage is applied, when the drain potentials of the nonvolatile memory transistors MT2′ of the pixel parts 100′ of the first line drop, the count values corresponding to the value of the lamp wave form voltage at that time are respectively held in the reading circuits 20′. The held count values are outputted from the output amplifier 60′ through the signal line 70′ under the control of the horizontal shift register 50′. After the count values are outputted, the transistor 20f′ is turned off to stop the application of the lamp wave form voltage and reset the count values. A similar driving operation is carried out after a second line to output second image pick-up signals (R signals and B signals) corresponding to the electric charges stored in the floating gates FG2′ of all lines.

After the second image pick-up signals are outputted, the control part 40′ draws out the electric charges stored in the floating gates FG1′ and FG2′ to the semiconductor substrate to erase the electric charges.

The above-described operations are carried out within one frame period. As described above, as the plurality of electric charge storage parts provided in the pixel parts, the nonvolatile memory transistors MT1′ and MT2′ are used so that the number of transistors may be reduced.

A structure that the electric charge storage part is formed with one transistor may be also applied to the pixel part shown in FIG. 3.

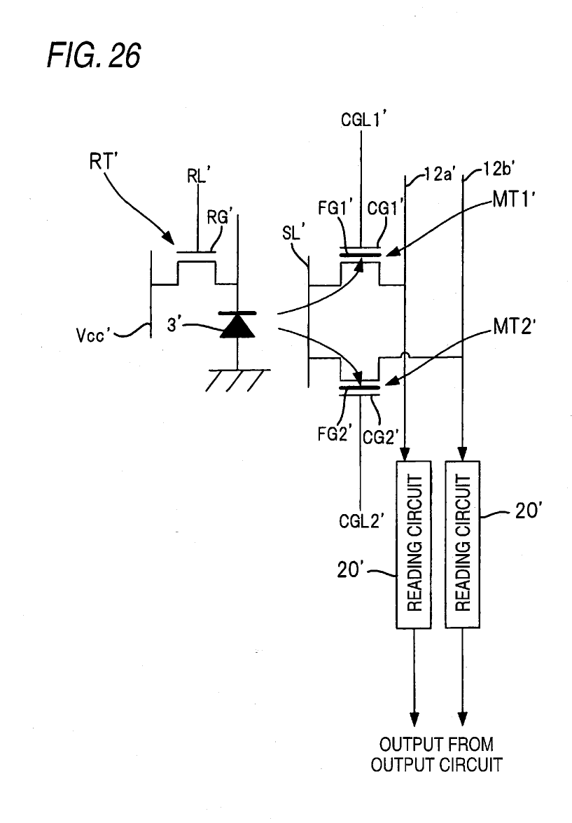

Further, in the example shown in FIG. 22, the nonvolatile memory transistor MT1′ and the nonvolatile memory transistor MT2′ are commonly connected to the one column signal line 12′ and the column signal line 12′ is connected to the one reading circuit 20′. However, as shown in FIG. 26, the nonvolatile memory transistor MT1′ and the nonvolatile memory transistor MT2′ may be respectively connected to separate column signal lines 12a′ and 12b′ and one reading circuits 20′ may be respectively connected to the column signal lines 12a′ and 12b′. Output circuits may be provided respectively correspondingly to the reading circuit 20′ connected to the column signal line 12a′ and the reading circuit 20′ connected to the column signal line 12b′. Thus, the first image pick-up signals and the second image pick-up signals may be simultaneously read outside the solid-state image pick-up element in parallel. As a result, a time from an image pick-up operation to an image displaying and recording operation may be shortened.

Further, in the solid-state image pick-up element having the pixel part shown in FIG. 3 or FIG. 18, the reading transistors of the electric charge storage parts may be respectively connected to separate signal lines and one reading circuits 20 may be respectively connected to the signal lines. Especially, in the case of the solid-state image pick-up element described in FIGS. 1 to 6, since the R signals, the G signals and the G signals may be simultaneously read in parallel, the image data may be formed at high speed.

Further, in the solid-state image pick-up element having the pixel part shown in FIG. 3 or FIG. 18, other area than the photoelectric conversion part 11 may be shielded by a light shield film and the photoelectric conversion part 11 may be extended to the lower parts of the channel areas of the writing transistors respectively. Thus, the electric injection efficiency may be improved.

As described above, below-described matters are disclosed in this specification.

The disclosed endoscope device includes: a light source that may independently emit a first light, a second light and a third light; and a solid-state image pick-up element having a plurality of pixel parts including a photoelectric conversion part that may receive the first light, the second light and the third light to generate electric charges corresponding to the received lights and a plurality of electric charge storage parts that may selectively store the electric charges generated in the photoelectric conversion part, and a signal reading part that independently reads signals corresponding to the electric charges respectively stored in the plurality of electric charge storage parts.

According to this structure, for instance, the first light, the second light and the third light are sequentially emitted. The first electric charges corresponding to the lights incident from the object to be shot or recorded in accordance with the first light are stored in one of the two electric charge storage parts of all the pixel parts. The second electric charges corresponding to the lights incident from the object to be shot or recorded in accordance with the second light are stored in the electric charge storage parts of the pixel parts half as many as all the pixel parts in which the first electric charges are not stored. The third electric charges corresponding to the lights incident from the object to be shot or recorded in accordance with the third light are stored in the electric charge storage parts of the remaining pixel parts half as many as all the pixel parts in which the first electric charges are not stored. Thus, the signals are read respectively from the electric charge storage parts so that the color image data may be formed. A usual structure needs to have steps of emitting a first light, storing electric charges, reading signals, emitting a second light, storing electric charges, reading signals, emitting a third light, storing electric charges, and reading signals. As compared therewith, the above-described structure may have the steps of emitting the first light, storing the electric charges, emitting the second light, storing the electric charges, emitting the third light, storing the electric charges, reading the signals corresponding to the first light, reading the signals corresponding to the second light and reading the signals corresponding to the third light. Accordingly, intervals of the exposure by color lights respectively may be shortened. Even when the object to be shot recorded moves, the color divergence may be prevented to improve an image quality.

In the disclosed endoscope device, the plurality of electric charge storage parts include a first electric charge storage part, a second electric charge storage part and a third electric charge storage part, and include a driving unit that carries out a first driving operation in which the first light is emitted to store in the first electric charge storage part the electric charge generated in the photoelectric conversion part by a light incident from an object to be shot or recorded relative to the first light, a second driving operation in which the second light is emitted to store in the second electric charge storage part the electric charge generated in the photoelectric conversion part by a light incident from an object to be shot or recorded relative to the second light and a third driving operation in which the third light is emitted to store in the third electric charge storage part the electric charge generated in the photoelectric conversion part by a light incident from an object to be shot or recorded relative to the third light. The signal reading part reads the signals corresponding to the electric charges respectively stored in the first electric charge storage part, the second electric charge storage part and the third electric charge storage part after the first driving operation, the second driving operation and the third driving operation are finished.

According to this structure, from the pixels parts respectively, the signals corresponding to the first light, the signals corresponding to the second light and the signals corresponding to the third light are obtained. Accordingly, when the first light, the second light and the third light are designated as primary colors (G, R, B) or complementary colors (Ye, Cy, Mg), the color image data may be formed. According to the above-described structure, since an interpolating process of a color signal is not necessary, false colors are decreased and a calculating time may be reduced.

In the disclosed endoscope device, the plurality of electric charge storage parts include a first electric charge storage part, a second electric charge storage part and a third electric charge storage part, the first light being a G light, the second light being a B light and the third light being an R light, and include a driving unit that carries out a first driving operation in which the G light, the B light and the R light are emitted at the same time or continuously to store in the first electric charge storage part the electric charge generated in the photoelectric conversion part by a light incident from an object to be shot or recorded relative to the emitted lights, a second driving operation in which the B light is emitted to store in the second electric charge storage part the electric charge generated in the photoelectric conversion part by a light incident from an object to be shot or recorded relative to the B light and a third driving operation in which the R light is emitted to store in the third electric charge storage part the electric charge generated in the photoelectric conversion part by a light incident from an object to be shot or recorded relative to the R light. The signal reading part reads the signals corresponding to the electric charges respectively stored in the first electric charge storage part, the second electric charge storage part and the third electric charge storage part after the first driving operation, the second driving operation and the third driving operation are finished, and includes a color difference signal generating unit that forms a first color difference signal from the signal read from the first electric charge storage part and the signal read from the second electric charge storage part and generates a second color difference signal from the signal read from the first electric charge storage part and the signal read from the third electric charge storage part.

According to this structure, from the pixel parts respectively, the signal (corresponding to the luminance signal Y) corresponding to the R light, the G light and the B light, the signal corresponding to the B light and the signal corresponding to the R light are obtained. Then, by these signals, the first color difference signal (corresponding to Cr, Pr) and the second color difference signal (corresponding to Cb, Pb) are obtained respectively for the pixel part. Accordingly, a time required for compressing the image data may be reduced. The luminance signal Y is ordinarily obtained by a calculation from the R signal, the G signal and the B signal. According to the above-described structure, when an amount of emission of the RGB lights is set on the basis of the coefficient for obtaining the luminance signal, the signal read from the first electric charge storage part corresponds to the luminance signal Y. Therefore, a calculating time until the image data is formed may be shortened and a frame rate at the time of shooting or recording a moving image may be improved.

In the disclosed endoscope device, the plurality of electric charge storage parts include a first electric charge storage part and a second electric charge storage part and include a driving unit that carries out a first driving operation in which the first light is emitted to store in the first electric charge storage parts of all the pixel parts the electric charges generated in the photoelectric conversion part by a light incident from an object to be shot or recorded relative to the first light, a second driving operation in which the second light is emitted to store in the second electric charge storage parts of the pixel parts half as many as the plurality of pixel parts the electric charges generated in the photoelectric conversion part by a light incident from an object to be shot or recorded relative to the second light and a third driving operation in which the third light is emitted to store in the second electric charge storage parts of the remaining pixel parts half as many as the plurality of pixel parts the electric charges generated in the photoelectric conversion part by a light incident from an object to be shot or recorded relative to the third light. The signal reading part reads the signals corresponding to the electric charges respectively stored in the first electric charge storage parts and the second electric charge storage parts after the first driving operation, the second driving operation and the third driving operation are finished and includes an interpolating unit that interpolates the signal corresponding to the second light or the signal corresponding to the third light that is not obtained from the pixel parts by using a signal corresponding to the second light and a signal corresponding to the third light that are obtained from pixel parts in the periphery of the pixel parts.

According to this structure, any of the signal corresponding to the first light, the signal corresponding to the second light and the signal corresponding to the third light is obtained from the pixel parts respectively. Then, the signal corresponding to the second light or the signal corresponding to the third light that is not obtained from the pixel parts is interpolated by using a signal corresponding to the second light and a signal corresponding to the third light that are obtained from pixel parts in the periphery of the pixel parts to form the signal corresponding to the first light, the signal corresponding to the second light and the signal corresponding to the third light for one pixel part. Accordingly, when the first light, the second light and the third light are designated as the primary colors (G, R, B) or the complementary colors (Ye, Cy, Mg), the color image data may be formed. Further, according to this structure, since the number of the electric charge storage parts may be two, the size of the pixel part may be reduced and the photoelectric conversion part may be enlarged to meet multi-pixels and a high sensitivity.

In the disclosed endoscope device, the plurality of electric charge storage parts further include a third electric charge storage part. The light source may further independently emit a fourth light. The driving unit also carries out a fourth driving operation in which the fourth light is emitted to store in the third electric charge storage parts of all the pixel parts the electric charges generated in the photoelectric conversion part by a light incident from an object to be shot or recorded relative to the fourth light. The signal reading part reads the signals corresponding to the electric charges respectively stored in the first electric charge storage parts, the second electric charge storage parts and the third electric charge storage parts after the first driving operation, the second driving operation, the third driving operation and the fourth driving operation are finished.

According to this structure, the signal corresponding to the fourth light is obtained from the pixel parts respectively. Accordingly, when the fourth light is designated as, for instance, an infrared ray, infrared imaged data in which a part hardly seen by the naked eye is emphasized may be formed. According to the above-described structure, since the electric charges necessary for forming the color image data and the infrared image data may be stored in short time, a difference in time for shooting or recording the color image data and the infrared image data may be decreased. As a result, for the same object to be shot or recorded, an image may be observed under different conditions and a proper diagnosis may be realized.

In the disclosed endoscope device, the plurality of electric charge storage parts further includes a third electric charge storage part. The light source may further independently emit a fourth light and a fifth light. The driving unit also carries out a fourth driving operation in which the fourth light is emitted to store in the third electric charge storage parts of the pixel parts half as many as the plurality of pixel parts the electric charges generated in the photoelectric conversion part by a light incident from an object to be shot or recorded relative to the fourth light and fifth driving operation in which the fifth light is emitted to store in the third electric charge storage parts of the remaining pixel parts half as many as the plurality of pixel parts the electric charges generated in the photoelectric conversion part by a light incident from an object to be shot or recorded relative to the fifth light. The signal reading part reads the signals corresponding to the electric charges respectively stored in the first electric charge storage parts, the second electric charge storage parts and the third electric charge storage parts after the first driving operation, the second driving operation, the third driving operation, the fourth driving operation and the fifth driving operation are finished.

According to this structure, the signals corresponding to the fourth light may be obtained from the pixel parts half as many as the plurality of pixel parts. Accordingly, when the fourth light is designated as, for instance, an infrared ray, infrared imaged data in which a part hardly seen by the naked eye is emphasized may be formed. Further, the signals corresponding to the fifth light may be obtained from the remaining pixel parts half as many as the plurality of pixel parts. Accordingly, when the fifth light is designated as, for instance, a light of a wavelength that may generate an excitation light from the object to be shot or recorded, excitation light image data may be formed in which cancer cells or the like are highlighted. According to the above-described structure, since the electric charges necessary for forming the color image data and the infrared image data may be stored in short time, a difference in time for shooting or recording the image data may be decreased. As a result, for the same object to be shot or recorded, an image may be observed under different conditions and a proper diagnosis may be realized.

In the disclosed endoscope device, the pixel parts respectively include electric charge discharge units that discharge the electric charges stored in the photoelectric conversion part to an external part before the light source emits the lights.

According to this structure, since the photoelectric conversion part is emptied before the lights are emitted, only the electric charges corresponding to the lights emitted thereafter may be stored. Thus, a color mixture may be prevented to improve an image quality.

In the disclosed endoscope device, the plurality of electric charge storage parts are respectively transistors including electric charge storage areas formed in upper parts of a semiconductor substrate on which the photoelectric conversion part is formed. The electric charges are stored in the electric charge storage areas. The signal reading part is formed with a reading circuit that reads, as the signals, the changes of the threshold voltages of the transistors respectively corresponding to the electric charges stored in the electric charge storage areas.

The disclosed endoscope device further includes a light shield film provided in the upper part of the semiconductor substrate and having an opening formed in an upper part of a part of the photoelectric conversion part.

The electric charge storage areas and channel areas of the transistors are covered with the light shield film and the photoelectric conversion part is extended to parts blow the channel areas of the transistors.

According to this structure, since the photoelectric conversion part is located below the channel areas of the transistors, the electric charges generated in the photoelectric conversion part in accordance with a light entering from the opening of the light shield film may be efficiently injected to the electric charge storage areas from the overlapped part of the photoelectric conversion part on the channel areas through the channel areas.

In the disclosed endoscope device, the electric charge storage area is a floating gate.

According to this structure, after the electric charges are stored in the floating gate, since the electric charges hardly receive an influence of noise from a periphery, an SN ratio may be improved.

In the disclosed endoscope device, the transistor includes two transistors of a writing transistor for injecting the electric charges to the floating gate and a reading transistor having a threshold voltage changed in accordance with the change of a potential of the floating gate to detect the threshold voltage. The writing transistor has a two-terminal structure including a source connected to the photoelectric conversion part and a gate.

According to this structure, a space for forming the drain of the writing transistor does not need to be provided in the pixel part. Thus, a layout in design may be improved to realize multi-pixels and a micronization.

In the endoscope device, the plurality of transistors included in the pixel parts are respectively connected to different output signal lines and the circuit is provided for the plurality of output signal lines respectively connected to the plurality of transistors.

According to this structure, the signals may be read in parallel from the plurality of transistors to carry out an image pick-up process at high speed.

In the disclosed endoscope device, the first light is the G light, the second light is the B light and the third light is the R light.

The disclosed method for driving an endoscope device includes a sold-state image pick-up element having a plurality of pixel parts. The plurality of pixel parts include a photoelectric conversion part that may receive lights incident from an object to be shot or recorded to generate electric charges corresponding to the received lights and a first electric charge storage part, a second electric charge storage part and a third electric charge storage part that may selectively store the electric charges generated in the photoelectric conversion part. The method for driving an endoscope device includes: a first driving step that emits a first light to store in the first electric charge storage part the electric charge generated in the photoelectric conversion part by the light incident from the object to be shot or recorded relative to the first light; a second driving step that emits a second light to store in the second electric charge storage part the electric charge generated in the photoelectric conversion part by the light incident from the object to be shot or recorded relative to the second light; a third driving step that emits a third light to store in the third electric charge storage part the electric charge generated in the photoelectric conversion part by the light incident from the object to be shot or recorded relative to the third light and a signal reading step that reads signals corresponding to the electric charges respectively stored in the first electric charge storage part, the second electric charge storage part and the third electric charge storage part after the first driving step, the second driving step and the third driving step are finished.

The disclosed method for driving an endoscope device includes a sold-state image pick-up element having a plurality of pixel parts. The plurality of pixel parts include a photoelectric conversion part that may receive lights incident from an object to be shot or recorded to generate electric charges corresponding to the received lights and a first electric charge storage part, a second electric charge storage part and a third electric charge storage part that may selectively store the electric charges generated in the photoelectric conversion part. The method for driving an endoscope device includes: a first driving step that emits a G light, a B light and an R light at the same time or continuously to store in the first electric charge storage part the electric charge generated in the photoelectric conversion part by the light incident from the object to be shot or recorded relative to the emitted lights; a second driving step that emits the B light to store in the second electric charge storage part the electric charge generated in the photoelectric conversion part by the light incident from the object to be shot or recorded relative to the B light; a third driving step that emits the R light to store in the third electric charge storage part the electric charge generated in the photoelectric conversion part by the light incident from the object to be shot or recorded relative to the R light; a signal reading step that reads signals corresponding to the electric charges respectively stored in the first electric charge storage part, the second electric charge storage part and the third electric charge storage part after the first driving step, the second driving step and the third driving step are finished; and a color difference signal generating step that forms a first color difference signal from the signal read from the first electric charge storage part and the signal read from the second electric charge storage part and generates a second color difference signal from the signal read from the first electric charge storage part and the signal read from the third electric charge storage part.

The method for driving an endoscope device includes a solid-state image pick-up element having a plurality of pixel parts. The plurality of pixel parts include a photoelectric conversion part that may receive lights incident from an object to be shot or recorded to generate electric charges corresponding to the received lights and a first electric charge storage part and a second electric charge storage part that may selectively store the electric charges generated in the photoelectric conversion part. The method for driving an endoscope device includes: a first driving step that emits a first light to store in the first electric charge storage parts of all the pixel parts the electric charges generated in the photoelectric conversion part by the light incident from the object to be shot or recorded relative to the first light; a second driving step that emits a second light to store in the second electric charge storage parts of the pixel parts half as many as the plurality of pixel parts the electric charges generated in the photoelectric conversion part by the light incident from the object to be shot or recorded relative to the second light; a third driving step that emits a third light to store in the second electric charge storage parts of the remaining pixel parts half as many as the plurality of pixel parts the electric charges generated in the photoelectric conversion part by the light incident from the object to be shot or recorded relative to the third light; a signal reading step that reads the signals corresponding to the electric charges respectively stored in the first electric charge storage parts and the second electric charge storage parts after the first driving step, the second driving step and the third driving step are finished and an interpolating step that interpolates the signal corresponding to the second light or the signal corresponding to the third light that is not obtained from the pixel parts by using a signal corresponding to the second light and a signal corresponding to the third light that are obtained from pixel parts in the periphery of the pixel parts.

In the disclosed method for driving an endoscope device, the pixel parts further include a third electric charge storage part. The method for driving an endoscope device includes: a fourth driving step that emits a fourth light to store in the third electric charge storage parts of all the pixel parts the electric charges generated in the photoelectric conversion part by the light incident from the object to be shot or recorded relative to the fourth light. The signal reading step sequentially reads the signals corresponding to the electric charges respectively stored in the first electric charge storage parts, the second electric charge storage parts and the third electric storage parts after the first driving step, the second driving step, the third driving step and the fourth driving step are finished.

In the disclosed method for driving an endoscope device, the pixel parts further include a third electric charge storage part. The method for driving an endoscope device includes: a fourth driving step that emits a fourth light to store in the third electric charge storage parts of the pixel parts half as many as the plurality of pixel parts the electric charges generated in the photoelectric conversion part by the light incident from the object to be shot or recorded relative to the fourth light; and a fifth driving step that emits a fifth light to store in the third electric charge storage parts of remaining pixel parts half as many as the plurality of pixel parts the electric charges generated in the photoelectric conversion part by the light incident from the object to be shot or recorded relative to the fifth light. The signal reading step sequentially reads the signals corresponding to the electric charges respectively stored in the first electric charge storage parts, the second electric charge storage parts and the third electric storage parts after the first driving step, the second driving step, the third driving step, the fourth driving step and the fifth driving step are finished.

The disclosed method for driving an endoscope device further includes: an electric charge discharge step that discharges the electric charges stored in the photoelectric conversion part to an external part before the lights are emitted.

In the disclosed method for driving an endoscope device, the first light is the G light, the second light is the B light and the third light is the R light.

What is claimed is:

1. An endoscope device comprising:
a light source that independently emits a first light, a second light and a third light; and
a solid-state image pick-up element including:
a plurality of pixel parts including: a photoelectric conversion part that receives the first light, the second light and the third light to generate electric charges corresponding to the received lights; and a plurality of electric charge storage parts that selectively stores the electric charges generated in the photoelectric conversion part; and
a signal reading part that independently reads signals corresponding to the electric charges respectively stored in the plurality of electric charge storage parts.

2. The endoscope device according to claim 1, wherein the plurality of electric charge storage parts include a first electric charge storage part, a second electric charge storage part and a third electric charge storage part, the plurality of electric charge storage parts drives:
a first driving operation in which the first light is emitted in
the first electric charge storage part for storing the electric charge generated in the photoelectric conversion part by a light incident from an object to be shot or recorded relative to the first light;
a second driving operation in which the second light is emitted in the second electric charge storage part for storing the electric charge generated in the photoelectric conversion part by a light incident from an object to be shot or recorded relative to the second light; and
a third driving operation in which the third light is emitted in the third electric charge storage part for storing the electric charge generated in the photoelectric conversion part by a light incident from an object to be shot or recorded relative to the third light, and
the signal reading part reads the signals corresponding to the electric charges respectively stored in the first electric charge storage part, the second electric charge storage part and the third electric charge storage part after the first driving operation, the second driving operation and the third driving operation are finished.

3. The endoscope device according to claim 1, further comprising:
a color difference signal generating unit in the solid-state image pick-up element,
wherein the plurality of electric charge storage parts include a first electric charge storage part, a second electric charge storage part and a third electric charge storage part, the first light being a G light, the second light being a B light and the third light being an R light,
the plurality of electric charge storage parts drives:
a first driving operation in which the G light, the B light and the R light are emitted at the same time or continuously to store in the first electric charge storage part for storing the electric charge generated in the photoelectric conversion part by a light incident from an object to be shot or recorded relative to the emitted lights;
a second driving operation in which the B light is emitted in the second electric charge storage part for storing the electric charge generated in the photoelectric conversion part by a light incident from an object to be shot or recorded relative to the B light; and
a third driving operation in which the R light is emitted in the third electric charge storage part for storing the electric charge generated in the photoelectric conversion part by a light incident from an object to be shot or recorded relative to the R light,
the signal reading part reads the signals corresponding to the electric charges respectively stored in the first electric charge storage part, the second electric charge storage part and the third electric charge storage part after the first driving operation, the second driving operation and the third driving operation are finished, and
the color difference signal generating unit generates a first color difference signal from the signal read from the first electric charge storage part and the signal read from the second electric charge storage part and generates a second color difference signal from the signal read from the first electric charge storage part and the signal read from the third electric charge storage part.

4. The endoscope device according to claim 1, further comprising:
an interpolating unit in the solid-state image pick-up element,
wherein the plurality of electric charge storage parts include a first electric charge storage part and a second electric charge storage part,
the plurality of electric charge storage parts drives:
a first driving operation in which the first light is emitted in the first electric charge storage parts of all the pixel parts for storing the electric charges generated in the photoelectric conversion part by a light incident from an object to be shot or recorded relative to the first light;
a second driving operation in which the second light is emitted in the second electric charge storage parts of the pixel parts half as many as the plurality of pixel parts for storing the electric charges generated in the photoelectric conversion part by a light incident from an object to be shot or recorded relative to the second light; and
a third driving operation in which the third light is emitted in the second electric charge storage parts of the remaining pixel parts half as many as the plurality of pixel parts for storing the electric charges generated in the photoelectric conversion part by a light incident from an object to be shot or recorded relative to the third light,
the signal reading part reads the signals corresponding to the electric charges respectively stored in the first electric charge storage parts and the second electric charge storage parts after the first driving operation, the second driving operation and the third driving operation are finished, and
the interpolating unit that interpolates the signal corresponding to the second light or the signal corresponding to the third light that is not obtained from the pixel parts by using a signal corresponding to the second light and a signal corresponding to the third light that are obtained from pixel parts in the periphery of the pixel parts.

5. The endoscope device according to claim 4, wherein the plurality of electric charge storage parts further include a third electric charge storage part,
the light source further independently emits a fourth light,
the plurality of electric charge storage parts drives a fourth driving operation in which the fourth light is emitted in the third electric charge storage parts of all the pixel parts for storing the electric charges generated in the photoelectric conversion part by a light incident from an object to be shot or recorded relative to the fourth light, and
the signal reading part reads the signals corresponding to the electric charges respectively stored in the first electric charge storage parts, the second electric charge storage parts and the third electric charge storage parts after the first driving operation, the second driving operation, the third driving operation and the fourth driving operation are finished.

6. The endoscope device according to claim 4, wherein the plurality of electric charge storage parts further include a third electric charge storage part, the light source further independently emits a fourth light and a fifth light,
the plurality of electric charge storage parts drives a fourth driving operation in which the fourth light is emitted in the third electric charge storage parts of the pixel parts half as many as the plurality of pixel parts for storing the electric charges generated in the photoelectric conversion part by a light incident from an object to be shot or recorded relative to the fourth light and a fifth driving operation in which the fifth light is emitted in the third electric charge storage parts of the remaining pixel parts half as many as the plurality of pixel parts for storing the electric charges generated in the photoelectric conversion part by a light incident from an object to be shot or recorded relative to the fifth light, and
the signal reading part reads the signals corresponding to the electric charges respectively stored in the first electric charge storage parts, the second electric charge storage parts and the third electric charge storage parts after the first driving operation, the second driving operation, the third driving operation, the fourth driving operation and the fifth driving operation are finished.

7. The endoscope device according to claim 1, further comprising:
an electric charge discharge unit in the pixel parts that discharges the electric charges stored in the photoelectric conversion part to an external part before the light source emits the lights.

8. The endoscope device according to claim 1, wherein the plurality of electric charge storage parts are respectively transistors including electric charge storage areas provided in upper parts of a semiconductor substrate on which the photoelectric conversion part is provided, the electric charges are stored in the electric charge storage areas and the signal reading part is provided with a reading circuit that reads, as the signals, the changes of the threshold voltages of the transistors respectively corresponding to the electric charges stored in the electric charge storage areas.

9. The endoscope device according to claim 8, further comprising:
a light shield film provided in the upper part of the semiconductor substrate and having an opening provided in an upper part of a part of the photoelectric conversion part, wherein the electric charge storage areas and channel areas of the transistors are covered with the light shield film and the photoelectric conversion part is extended to parts blow the channel areas of the transistors.

10. The endoscope device according to claim 8, wherein the electric charge storage area is a floating gate.

11. The endoscope device according to claim 10, wherein the transistor includes two transistors of a writing transistor for injecting the electric charges to the floating gate and a reading transistor having a threshold voltage changed in accordance with the change of a potential of the floating gate to detect the threshold voltage and the writing transistor has a two-terminal structure including a source connected to the photoelectric conversion part and a gate.

12. The endoscope device according to claim 8, wherein the plurality of transistors included in the pixel parts are respectively connected to different output signal lines and the circuit is provided for the plurality of output signal lines respectively connected to the plurality of transistors.

13. The endoscope device according to claim 1, wherein the first light is the G light, the second light is the B light and the third light is the R light.

14. A method for driving an endoscope device including a sold-state image pick-up element having a plurality of pixel parts, the plurality of pixel parts including a photoelectric conversion part that receive lights incident from an object to be shot or recorded to generate electric charges corresponding to the received lights and a first electric charge storage part, a second electric charge storage part and a third electric charge storage part that selectively store the electric charges generated in the photoelectric conversion part, the method for driving an endoscope device comprising:
firstly driving to store the electric charge, which is generated in the photoelectric conversion part by a light incident from the object to be shot or recorded relative to a first light, in the first electric charge storage part after the first light is emitted;
secondly driving to store the electric charge, which is generated in the photoelectric conversion part by a light incident from the object to be shot or recorded relative to a second light, in the second electric charge storage part after the second light is emitted;
thirdly driving to store the electric charge, which is generated in the photoelectric conversion part by a light incident from the object to be shot or recorded relative to a third light, in the third electric charge storage part after the third light is emitted; and
reading signals corresponding to the electric charges respectively stored in the first electric charge storage part, the second electric charge storage part and the third electric charge storage part after the first driving, the second driving and the third driving are finished.

15. A method for driving an endoscope device including a sold-state image pick-up element having a plurality of pixel parts, the plurality of pixel parts including a photoelectric conversion part that receive lights incident from an object to be shot or recorded to generate electric charges corresponding to the received lights and a first electric charge storage part, a second electric charge storage part and a third electric charge storage part that selectively store the electric charges generated in the photoelectric conversion part, the method for driving an endoscope device comprising:
firstly driving to store the electric charge, which is generated in the photoelectric conversion part by a light incident from the object to be shot or recorded relative to a G light, a B light and an R light lights, in the first electric charge storage part after the G light, the B light and the R light at the same time or continuously are emitted;
secondly driving to store the electric charge, which is generated in the photoelectric conversion part by a light incident from the object to be shot or recorded relative to the B light, in the second electric charge storage part after the B light is emitted;
thirdly driving to store the electric charge, which is generated in the photoelectric conversion part by a light incident from the object to be shot or recorded relative to the R light, in the third electric charge storage part after the R light is emitted;
a signal reading reads signals corresponding to the electric charges respectively stored in the first electric charge storage part, the second electric charge storage part and the third electric charge storage part after the first driving, the second driving and the third driving are finished; and
generating a first color difference signal from the signal read from the first electric charge storage part and the signal read from the second electric charge storage part and generates a second color difference signal from the signal read from the first electric charge storage part and the signal read from the third electric charge storage part.

16. A method for driving an endoscope device including a solid-state image pick-up element having a plurality of pixel parts, the plurality of pixel parts including a photoelectric conversion part that receive lights incident from an object to be shot or recorded to generate electric charges corresponding to the received lights and a first electric charge storage part and a second electric charge storage part that selectively store the electric charges generated in the photoelectric conversion part, the method for driving an endoscope device comprising:
firstly driving to store the electric charges, which is generated in the photoelectric conversion part by a light incident from the object to be shot or recorded relative to a first light, in the first electric charge storage part of all the pixel parts after the first light is emitted;
secondly driving to store the electric charges, which is generated in the photoelectric conversion part by a light incident from the object to be shot or recorded relative to a second light, in the second electric charge storage parts of the pixel parts half as many as the plurality of pixel parts after the second light is emitted;
thirdly driving to store the electric charges, which is generated in the photoelectric conversion part by a light incident from the object to be shot or recorded relative to a third light, in the second electric charge storage parts of the remaining pixel parts half as many as the plurality of pixel parts after the third light is emitted;
reading the signals corresponding to the electric charges respectively stored in the first electric charge storage parts and the second electric charge storage parts after the first driving, the second driving and the third driving are finished; and
interpolating the signal corresponding to the second light or the signal corresponding to the third light that is not obtained from the pixel parts by using a signal corresponding to the second light and a signal corresponding to the third light that are obtained from pixel parts in the periphery of the pixel parts.

17. The method for driving an endoscope device according to claim 16, the pixel parts further including a third electric charge storage part, the method for driving an endoscope device comprising:
fourthly driving to store the electric charges, which is generated in the photoelectric conversion part by a light incident from the object to be shot or recorded relative to a fourth light, in the third electric charge storage parts of all the pixel parts after the fourth light is emitted,
wherein the reading reads the signals corresponding to the electric charges respectively stored in the first electric charge storage parts, the second electric charge storage parts and the third electric storage parts after the first driving, the second driving, the third driving and the fourth driving are finished.

18. The method for driving an endoscope device according to claim 16, the pixel parts further including a third electric charge storage part, the method for driving an endoscope device comprising:
fourthly driving to store the electric charges, which is generated in the photoelectric conversion part by a light incident from the object to be shot or recorded relative to a fourth light, in the third electric charge storage parts of the pixel parts half as many as the plurality of pixel parts after the fourth light is emitted; and
fifthly driving to store the electric charges, which is generated in the photoelectric conversion part by a light incident from the object to be shot or recorded relative to a fifth light, in the third electric charge storage parts of remaining pixel parts half as many as the plurality of pixel parts after the fifth light is emitted;
wherein the signal sequentially reading reads the signals corresponding to the electric charges respectively stored in the first electric charge storage parts, the second electric charge storage parts and the third electric storage parts after the first driving, the second driving, the third driving, the fourth driving and the fifth driving are finished.

19. The method for driving an endoscope device according to claim 14, further comprising:
discharging the electric charges stored in the photoelectric conversion part to an external part before the lights are emitted.

20. The method for driving an endoscope device according to claim 14, wherein the first light is the G light, the second light is the B light and the third light is the R light.
